(19) United States
(12) Patent Application Publication
(10) Pub. No.: US 2025/0313544 A1
(43) Pub. Date: Oct. 9, 2025

(54) INHIBITORS OF GLUTATHIONE S-TRANSFERASE ZETA 1 (GSTZ1) AND METHODS OF USE

(71) Applicant: H. LEE MOFFITT CANCER CENTER AND RESEARCH INSTITUTE, INC., Tampa, FL (US)

(72) Inventors: Andrii MONASTYRSKYI, Lutz, FL (US); Uwe RIX, Tampa, FL (US); Yi LIAO, Lutz, FL (US); Sean CHIN CHAN, Tampa, FL (US)

(21) Appl. No.: 18/864,680

(22) PCT Filed: May 10, 2023
(86) PCT No.: PCT/US2023/021681

§ 371 Date: Nov. 11, 2024

Related U.S. Application Data
(60) Provisional application No. 63/340,245, filed on May 10, 2022.

(51) Int. Cl. C07D 401/12; A61K 31/498; C07D 241/44; C07D 403/12

(52) U.S. Cl. CPC C07D 401/12; A61K 31/498; C07D 241/44; C07D 403/12

(57) ABSTRACT

The present disclosure provides compounds useful in treating medical disorders, more particular inhibitors of glutathione S-transferase zeta 1 (GSTZ1) which are useful in treating cancers in subjects in need thereof. Compositions comprising said compounds and methods of making and using said compounds are also provided.

### CROSS-REFERENCE TO RELATED APPLICATIONS

This application claims the benefit of priority to U.S. Provisional Application No. 63/340,245, filed May 10, 2022, the disclosure of which is incorporated herein by reference in its entirety.

### TECHNICAL FIELD

This disclosure relates to compounds useful in treating medical disorders, and more particularly to inhibitors of GSTZ1 useful in treating cancers.

### BACKGROUND

Oncogenic alterations are common hallmarks of cancer, and using small-molecule inhibitors to target these alterations is effective in cancer management and treatment. In non-small cell lung cancer (NSCLC), KRAS G12C is the most frequent KRAS mutation in lung adenocarcinoma (LUAD), but only 30-50% of patients harboring KRAS G12C respond to the targeted therapy such as sotorasib. In squamous cell lung cancer (LUSQ), KRAS mutations are rarely detected, but other oncogenic aberrations such as FGFR1 amplification (˜20%) and DDR2 mutation (˜4%) have been described and associated with cancer cell vulnerability towards drugs that target these tyrosine (Tyr) kinases. However, the efficacy of single agent infigratinib (a pan-FGFR inhibitor) and dasatinib (a DDR2 inhibitor) was limited in clinic trials. There are no targeted therapies approved for treating LUSQ. Although there is a deep biological understanding of these oncogenic aberrations, little is known about intrinsic resistance mechanisms underlying oncogenic alteration inhibition by these targeted drugs in lung cancer.

It is well accepted that GSTs family enzymes are attractive anti-cancer targets given their general cytoprotective roles in cancer cell survival and drug resistance, and targeting GSTs and impairing redox balance have emerged as an effective strategy for developing novel anti-cancer therapeutics. Similar to other GST family enzymes, GSTZ1 was also known to modulate the activity of anti-cancer drugs and participate in multiple biological processes, including metabolic and redox homeostasis. Despite that, GSTZ1 has rarely been studied in NSCLCs with oncogenic alterations and has no selective or potent inhibitors. This disclosure addresses this as well as other needs.

### SUMMARY

The present disclosure provides compounds useful in treating medical disorders, more particular inhibitors of glutathione S-transferase zeta 1 (GSTZ1) which are useful in treating cancers in subjects in need thereof. Compositions comprising said compounds and methods of making and using said compounds are also provided.

In one aspect, a compound is provided of Formula I

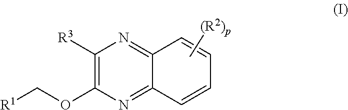

or a pharmaceutical acceptable salt thereof, wherein all variables are as defined herein.

In another aspect, a pharmaceutical composition is provided comprising a compound described herein, or a pharmaceutically acceptable salt, in combination with a pharmaceutically acceptable carrier or excipient.

In another aspect, a method of treating a cancer in a subject in need thereof is provided, the method comprising administering a therapeutically effective amount of a compound described herein, or a pharmaceutically acceptable salt thereof, or a pharmaceutical composition described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 provides the design and synthesis of fully functionalized fragment-like BioCore and BioCore-like probes. Structural features showing fragment-probes composed of a BioCore(-like) scaffold, a diazirine moiety, and an alkyne handle.

FIGS. 2A-2C provide the exploration of the druggable proteome in live H1792 lung cancer cells using fragment probes. FIG. 2A) Workflow of mass spectrometry-based chemical proteomics coupled with label-free quantitation. FIG. 2B) Pie chart showing the number of proteins enriched by each probe based on log 2 ratio vs. control probe of >2.32 and p<0.05 from t-test. FIG. 2C) Scatter plot displaying the distribution of probe-enriched proteins based on the log 2 ratio vs. control probe and the cross-comparison enrichment score, which is the difference between the average of a protein signal intensity for a given 30 probe and the average of that protein signal intensity across all other probes+standard deviation (SD). Dashed red lines indicate cutoffs of 2 for enrichment score cross-comparison and of 2.32 (i.e. 5-fold enrichment over control probe) for log 2 ratio. Black box shows proteins for which log 2 ratios cannot be accurately computed due to lack of identification by the control probe. High-confidence targets enriched with higher selectivity by individual probes are displayed by red dots (box with red background). Proteins in box with yellow background are potential target candidates that are more commonly shared across different probes.

FIGS. 3A-3F provide the validation of the interaction between probe 17 and GSTZ1. FIG. 3A) Scheme displaying target validation approaches with either in-gel fluorescence imaging or immunoblotting (WB) imaging. Probe crosslinking was conducted in live cells. FIG. 3B) Design and chemical structure of probe 17 and its competitor. FIG. 3C) In-gel fluorescence analysis of probe-labeled proteins. FIG. 3D) WB imaging of GSTZ1 pulldown and competition (compt). TCL: total cell lysate. FIG. 3E) The BioCore of probe 17 was docked to the GSH-binding pocket of GSTZ1 (PDB: 1FW1) using Glide. Nitrogen and oxygen atoms of amino acids were colored in blue and red, respectively. Hydrogen bond formed with Cys 16 or Gln 111 was colored in red. A green molecule represents the GSH. FIG. 3F) GSTZ1 enzymatic activity inhibition by probe 17 at the dose of 500 M and at the time of 10 mins.

FIGS. 4A-4F provide GSTZ1 expression levels in lung cancer patients and cell lines. FIGS. 4A and 4B) Differential GSTZ1 gene expression levels between TCGA normal tissues and lung tumors of the LUAD (4A) and LUSQ (4B) subtypes. FIGS. 4C and 4D) Kaplan-Meier survival analysis against 719 LUAD patients (4C) and 130 LUSQ patients (4D). FIG. 4E) DepMap-based GSTZ1 gene dependency extracted from the combined RNAi database (Achilles+DRIVE+Marcotte) in relation to the oncogene dependency for DDR2, FGFR1 and KRAS. The lower the gene effect score, the more essential a gene is. FIG. 4F) GSTZ1 protein levels in lung fibroblasts and drug-refractory NSCLC cell lines.

FIGS. 5A-5D provide the effect of GSTZ1 knockdown on cell viability in response to clinical drugs. SMARTPool targeted siGSTZ1 (25 nM) was used to knock down GSTZ1 expression in H1792 (FIG. 5A), H520 (FIG. 5B), HCC366 (FIG. 5C), H2286 (FIG. 5D) cells. Cells were incubated with siGSTZ1 for 24 h, followed by the treatment of indicated drugs for an additional 72 h. After harvesting cells, a small aliquot of cells was stained, and viable cells were counted using the trypan blue assay. Unstained cells were subjected to western blotting to determine GSTZ1 knockdown efficiency. One-way ANOVA was applied for statistical analysis and asterisks represent *: p-value<0.05; **: p-value<0.01; ***: p-value<0.001; n.s.: not significant; NT: non-targeting siRNA.

FIGS. 6A-6F provide that the combined sotorasib treatment and GSTZ1 knockdown disrupts proteomic Tyr reactivity. FIG. 6A) Chemical features of sulfur-triazole exchange (SuTEx) probe HHS-482 and tyrosine labeling reaction. FIG. 6B) Pathway analysis of significantly altered proteins upon GSTZ1 knockdown in H1792 cells. FIGS. 6C and 6D) Western blot of oncogene expression and tyrosine phosphorylation of KRAS^{G12C} in H1792 (6C) and FGFR1 in H520 (6D) cells. One-way ANOVA was applied for statistical analysis. **: p-value<0.01; ***: p-value<0.001. n.s.: not significant. FIGS. 6E and 6F) Western blot of apoptosis markers cleaved (c-) PARP1 and caspase 3 induced by siGSTZ1 and/or clinical drugs. Soto: sotorasib; Infig: infigratinib.

FIGS. 7A-7I provide high-confidence targets of selected probes. FIG. 7A) Synthetic scheme for the preparation of 22 probes, which follows the synthesis described by Parker et al. with the exception of generation of compound 29 from compound 28. FIGS. 7B, 7C, and 7D) Cutoffs of log 2 ratio>2.32 and enrichment score>2 were applied. Boxed are proteins that log 2 ratio cannot be computed for due to missing values in the control group. FIG. 7E) Target enrichment plot of probe 17 over control probe 22. Blue lines indicate cutoffs: log 2 ratio=2.32 (as in FIG. 2C) and −log p=1.3 (i.e. p 0.05). Boxes indicate proteins for which log 2 ratio cannot be computed due to missing values in the control group. FIGS. 7F and 7G) The enrichment pattern of GSTZ1 (7F) and MMGT1 (7G) across all probes. Boxes indicate probes for which the respective target was not detected. FIGS. 7H and 7I) In-gel fluorescence pattern of probe 5-(7H) and probe 21-(7I) labeled proteins by following the fluorescent imaging approach. UT represents the vehicle treatment with DMSO. TCL indicates total cell lysates.

FIGS. 8A-8E provide the molecular interaction of probe 17 with GSTZ1. Molecular modeling of probe 17 (yellow sticks) docked to the GSH-binding pocket of GSTZ1 (FIG. 8A), overlay of probe 17 and GSH (green sticks) (FIG. 8B) and overlay of probe 17 and corresponding BioCore of probe 17 (orange sticks) (FIG. 8C). (FIG. 8D) GSTZ1 enzymatic activity inhibition by probe 17. GSTZ1-catalyzed reaction was monitored by spectrophotometry. Reaction was run at room temperature for 10 minutes. ‘BLK’ represents buffer only. (FIG. 8E) Inhibition of GSTZ1 enzymatic activity by probe 17 upon UV irradiation and at 10 mins. ‘UT’ indicates DMSO-treated samples.

FIGS. 9A-9F provide the cellular activity of probes and/or clinical drugs in drug-refractory NSCLC cells. Cellular activity of 22 fragment-like probes was evaluated in (FIG. 9A) H1792 cells harboring KRAS G12C; (FIG. 9B) H520 cells harboring amplified FGFR1; (FIG. 9C) HCC366 cells harboring mutated DDR2. Probe 17 was combined with sotorasib (Soto) in (FIG. 9D) H1792 cells at the dose of 50 μM; (FIG. 9E) Infigratinib (Infig) in H520 cells at the dose of 5 μM; (FIG. 9F) Dasatinib (Dasa) in HCC366 cells at the dose of 10 μM. Cell viability was measured using the CellTiter-Glo assay. One-way ANOVA was applied for statistical analysis and asterisks represent *: p-value<0.05; **: p-value<0.01.

FIGS. 10A-10H provide protein labeling by SuTEx. (FIG. 10A) H1792 cells were treated with SMARTPool targeted siGSTZ1 (25 nM) for 72 h and then treated with Sotorasib (Soto) for additional 24 h. Cell lysates were labeled with HHS-482 (25 μM) for 1 h. Labeled proteome was visualized using in-gel fluorescence and then subjected to western blotting against GSTZ1 and GAPDH. UT: non-targeting siRNA+DMSO. (FIG. 10B) In-gel fluorescence imaging of SuTEx-labeled H1792 proteome treated with vehicle/DMSO (UT), siGSTZ1, Soto, and Soto+siGSTZ1 combination. (FIG. 10C) Western blot of GSTZ1 expression in indicated samples. NT: non-targeting siRNA. (FIGS. 10D, 10E, and 10F) LC-MS/MS analysis of the proteome-wide Tyr reactivity alterations induced by treatment with siGSTZ1 (10D), Soto (10E), Soto+siGSTZ1 combination (10F) in H1792 cells. Comparison was made with vehicle-treated samples (UT) and one-sample t test was performed. Cutoffs were applied as follows: log 2 ratio≤−0.585 or ≥0.585, p value<0.05, and significantly altered proteins were highlighted in red. (FIGS. 10G and 10H) Pathway analysis of significantly altered proteins. Significantly altered proteins induced by Soto (10G) and Soto+siGSTZ1 combination (10H) were used as input for pathway analysis. Results from Reactome analysis were colored in red and some are known to mediate resistance to Soto. Results from hallmark gene set analysis were colored in blue. EMT: epithelial-mesenchymal transition; RTKs: receptor tyrosine kinases; GFRs: growth factor receptors; AAs: amino acids.

FIGS. 11A-11B provide immunoblot analysis of modulation of tyrosine phosphorylation by GSTZ1. Western blot of GSTZ1 expression and global tyrosine phosphorylation in H1792 (FIG. 11A) and H520 cells (FIG. 11B), respectively. Red arrows were pointed at visually observable phosphorylation alterations across 4 treatments.

FIGS. 12A-12D provide PRTC-based normalization of fragment-chemoproteomics mass spectrometry data. Sum of PRTC precursor ion signals across 44 MS runs before normalization (FIG. 12A) vs. after normalization (FIG. 12B). (FIGS. 12C and 12D) PRTC-based normalization of SuTEx mass spectrometry data. Sum of PRTC precursor ion signals across 16 MS runs before normalization (12C) vs. after normalization (12D).

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain, benefiting from the teachings presented in the descriptions herein and the associated drawings. Therefore, it is understood that the disclosures are not limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual embodiments described and illustrated herein has discrete components and features that may be readily separated from or combined with the features of any of the other several embodiments without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise. Thus, for example, reference to “a compound,” “a composition,” or “a cancer” includes, but is not limited to, two or more such compounds, compositions, or cancers, and the like.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single dose compositions can contain such amounts or submultiples thereof to make up the daily dose. The individual physician can adjust the dosage in the event of any contraindications. It is generally preferred that a maximum dose of the pharmacological agents of the invention (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition such as a cancer. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some embodiments, the alkyl group is optionally substituted as described herein. The term “alkyl” as used herein is not intended to be limited to monovalent radicals and may include polyvalent radical groups as appropriate, such as divalent, trivalent, tetravalent, pentavalent, and hexavalent alkyl, and the like, based on the position and location of such groups in the compounds described herein as would be readily understood by the skilled person.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some embodiments, the cycloalkyl group is optionally substituted as described herein. The term “cycloalkyl” as used herein is not intended to be limited to monovalent radicals and may include polyvalent radical groups as appropriate, such as divalent, trivalent, tetravalent, pentavalent, and hexavalent cycloalkyl, and the like, based on the position and location of such groups in the compounds described herein as would be readily understood by the skilled person.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein. The term “alkenyl” as used herein is not intended to be limited to monovalent radicals and may include polyvalent radical groups as appropriate, such as divalent, trivalent, tetravalent, pentavalent, and hexavalent alkenyl, and the like, based on the position and location of such groups in the compounds described herein as would be readily understood by the skilled person.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein. The term “alkynyl” as used herein is not intended to be limited to monovalent radicals and may include polyvalent radical groups as appropriate, such as divalent, trivalent, tetravalent, pentavalent, and hexavalent alkynyl, and the like, based on the position and location of such groups in the compounds described herein as would be readily understood by the skilled person.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—).

“Alkanoyl” is an alkyl group as defined above covalently bound through a carbonyl (C═O) bridge. The carbonyl carbon is included in the number of carbons, for example C₂alkanoyl is a CH₃(C═O)-group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein. The term “aryl” as used herein is not intended to be limited to monovalent radicals and may include polyvalent radical groups as appropriate, such as divalent, trivalent, tetravalent, pentavalent, and hexavalent aryl, and the like, based on the position and location of such groups in the compounds described herein as would be readily understood by the skilled person.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S-portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo|1,4|dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. The term “heterocycle” as used herein is not intended to be limited to monovalent radicals and may include polyvalent radical groups as appropriate, such as divalent, trivalent, tetravalent, pentavalent, and hexavalent heterocycle, and the like, based on the position and location of such groups in the compounds described herein as would be readily understood by the skilled person.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one embodiment, the total number of S and O atoms in the heteroaryl ring is not more than 2. In another embodiment, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The term “heteroaryl” as used herein is not intended to be limited to monovalent radicals and may include polyvalent radical groups as appropriate, such as divalent, trivalent, tetravalent, pentavalent, and hexavalent heteroaryl, and the like, based on the position and location of such groups in the compounds described herein as would be readily understood by the skilled person.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Suitable counterions found in pharmaceutically acceptable salts described herein include, but are not limited to, cations such as calcium, chloroprocaine, choline, diethanolamine, ethanolamine, ethylenediamine, meglumine, potassium, procaine, sodium, triethylamine, and zinc, and anions such as acetate, aspartate, benzenesulfonate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, and tosylate. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

The present disclosure provides compounds which are useful as inhibitor of glutathione S-transferase zeta 1 (GSTZ1) which are useful in the treatment of medical disorders, such as cancers.

Thus, in one aspect, a compound is provided of Formula I

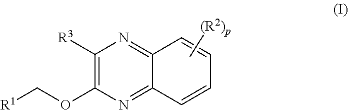

or a pharmaceutically acceptable salt thereof;
wherein:
R¹ is selected from 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more groups independently selected from X as allowed by valency;

R² is independently selected at each occurrence from hydrogen, halo, nitro, cyano, C₁-C₆ alkyl, C₁-C₆ haloalkyl, R^{x}O—(C₀-C₃ alkyl)-, (R^{x}R^{y}N)—(C₀-C₃ alkyl)-, R^{x}O—C(O)—(C₀-C₃ alkyl)-, (R^{x}R^{y}N)—C(O)—(C₀-C₃ alkyl)-, R^{x}O—S(O)₂—(C₀-C₃ alkyl)-, (R^{x}R^{y}N)—S(O)₂—(C₀-C₃ alkyl)-, R^{z}C(O)—(C₀-C₆ alkyl)-, and R^{z}S(O)₂—(C₀-C₃ alkyl)-;
p is 1, 2, 3, or 4;
R³ is selected from C₁-C₆ alkyl, C₁-C₆ haloalkyl, (C₃-C₆ cycloalkyl) (C₀-C₃ alkyl)-, and R^{x}O—(C₀-C₃ alkyl)-;
X is independent selected at each occurrence from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, (R^{x}R^{y}N)—(C₀-C₃ alkyl)-, R^{x}O—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—(R^{x}N)—(C₀-C₃ alkyl)-, R^{z}C(O)—(C₀-C₆ alkyl)-, and R^{z}S(O)₂-(C₀-C₃ alkyl)-, each of which may be optionally substituted with one or more groups independently selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more groups independently selected from Y as allowed by valency;
R^{z} is independently selected at each occurrence from hydrogen, halo, C₁-C₀alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more groups independently selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects of Formula I, R¹ is 3- to 8-membered monocyclic or bicyclic heterocycle optionally substituted with one or more groups independently selected from X as allowed by valency. 3. In some aspects of Formula I, R¹ is selected from:

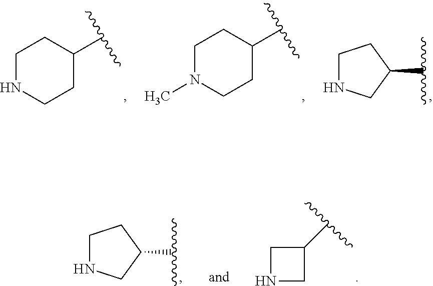

In some aspects of Formula I, R¹ is 6- to 10-membered monocyclic or bicyclic aryl optionally substituted with one or more groups independently selected from X as allowed by valency. In some aspects of Formula I, R¹ is

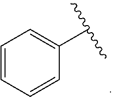 [SMILES: *c1ccccc1]

In some aspects of Formula I, R¹ is 5- to 10-membered monocyclic or bicyclic heteroaryl. In some aspects of Formula I, R¹ is selected from:

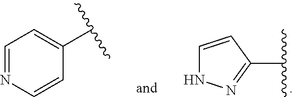 [SMILES: *c1ccncc1 ; *c1cc[nH]n1]

In some aspects of Formula I, R³ is C₁-C₆ alkyl. In some aspects of Formula I, R³ is selected from methyl, ethyl, isopropyl, and tert-butyl.

In some aspects of Formula I, R³ is C₁-C₆ haloalkyl. In some aspects of Formula I, R³ is selected from —CF₃ and —CH₂CF₃.

In some aspects of Formula I, R³ is (C₃-C₆ cycloalkyl) (C₀-C₃ alkyl)-. In some aspects of Formula I, R³ is selected from:

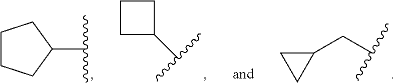

In some aspects of Formula I, R³ is R^{x}O—(C₀-C₃ alkyl)-. In some aspects of Formula I, R³ is —CH₂OCH₃.

In some aspects of Formula I, p is 1. In some aspects of Formula I, p is 2. In some aspects of Formula I, p is 3. In some aspects of Formula I, p is 4.

In some aspects of Formula I, R² is independently selected at each occurrence from hydrogen, chloro, bromo, iodo, —OH, —OCH₃, —CH₃, tert-butyl, —CF₃, —NH₂, —N(CH₃)₂, nitro, cyano, —S(O)₂—CH₃, —S(O)₂—NH₂, and —C(O)—NH₂.

In some aspects of Formula I,

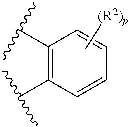

is selected from:

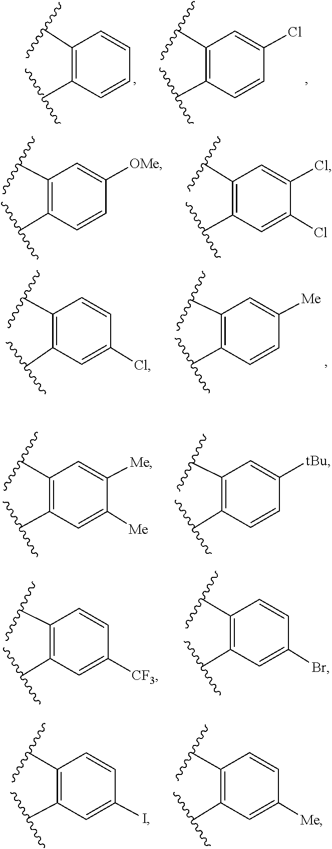

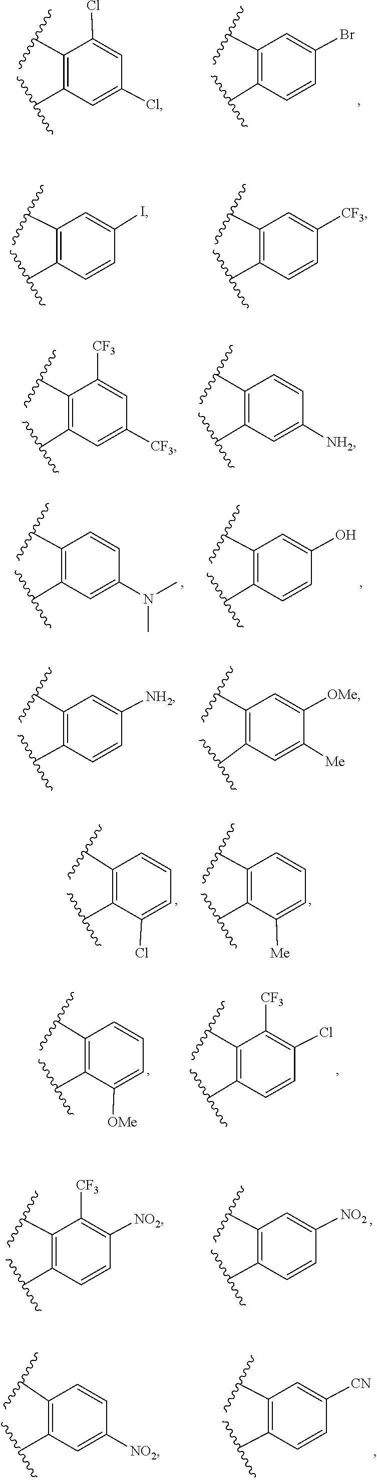

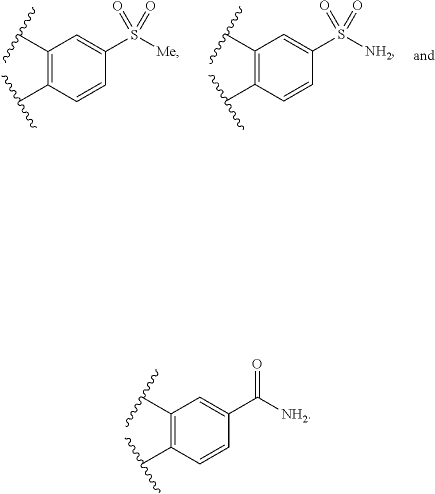 [SMILES: *c1ccc(S(C)(=O)=O)cc1* ; *c1ccc(S(N)(=O)=O)cc1* ; *c1ccc(C(N)=O)cc1*]

In another aspect, a compound is provided selected from:

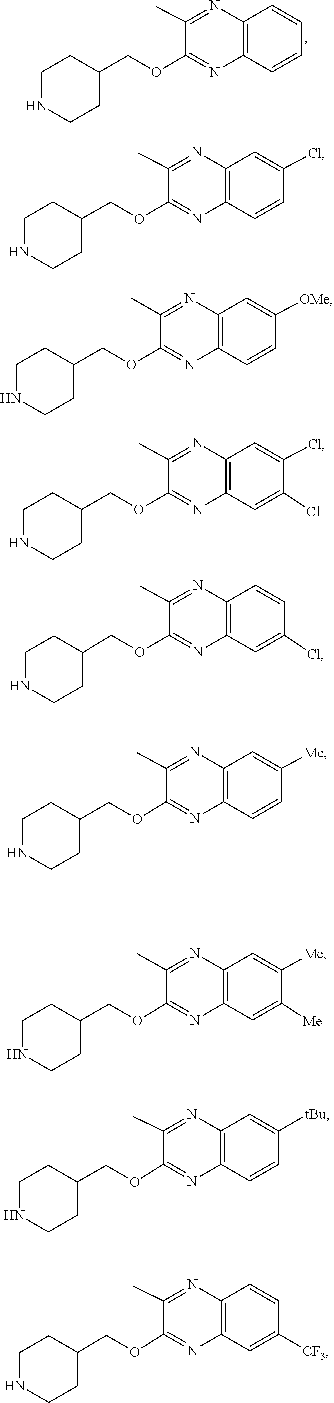

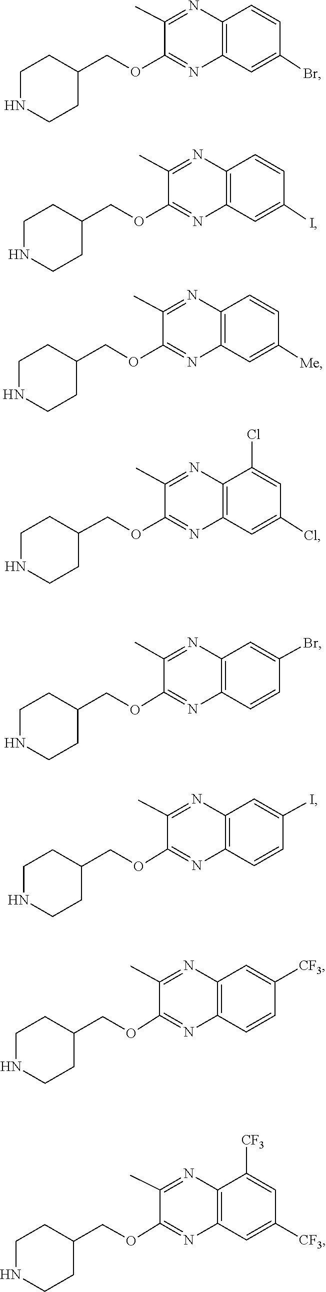

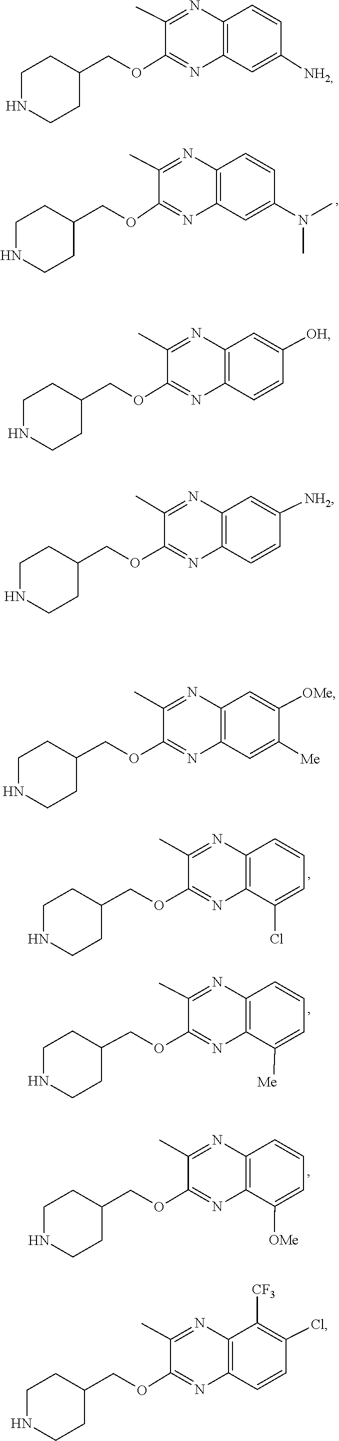

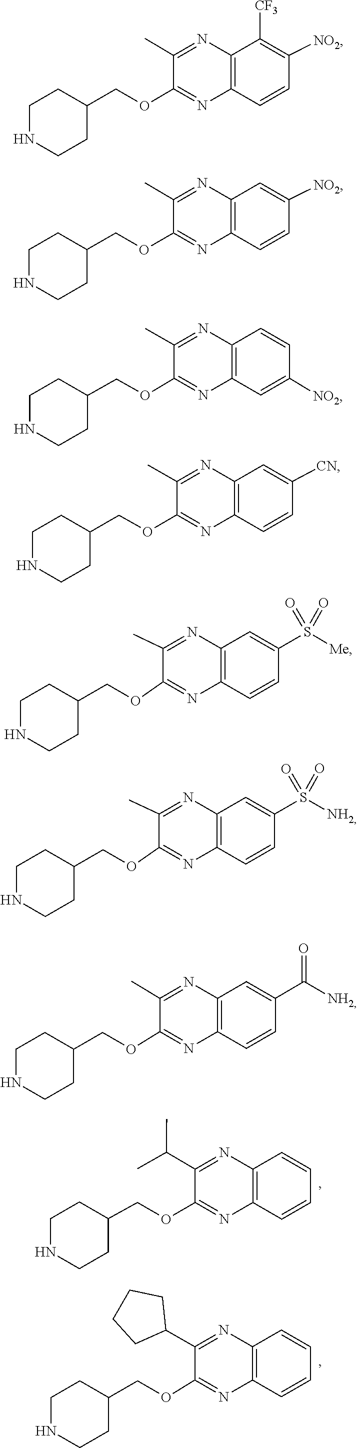

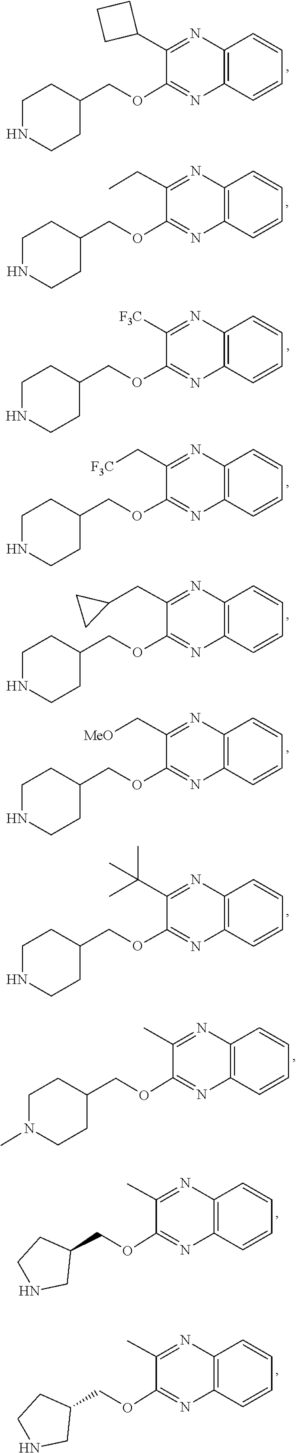

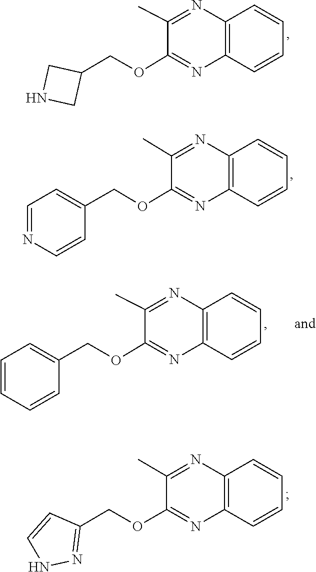 [SMILES: Cc1nc2ccccc2nc1OCC1CNC1 ; Cc1nc2ccccc2nc1OCc1ccncc1 ; Cc1nc2ccccc2nc1OCc1ccccc1 ; Cc1nc2ccccc2nc1OCc1cc[nH]n1]

or a pharmaceutically acceptable salt thereof.

The present disclosure also includes compounds of Formula I with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### Methods of Treatment

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some embodiments, the compounds can inhibit GSTZ1. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas. Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors. The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDS-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some embodiments, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary and can be administered in one or more dose administrations daily, for one or several days.

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some embodiments, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth to provide those of ordinary skill in the art with a complete disclosure and description of how the compounds, compositions, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

Chemical Proteomics with Novel Fully Functionalized Fragments and Stringent Target Prioritization Identifies the Glutathione-Dependent Isomerase GSTZ1 as a Lung Cancer Target

Photoreactive fragment-like probes have been applied to discover target proteins that constitute novel cellular vulnerabilities and to identify viable chemical hits for drug discovery. Through forming covalent bonds, functionalized probes can achieve stronger target engagement and require less effort for on-target mechanism validation. However, the design of probe libraries, which directly affects the biological target space that is interrogated, and effective target prioritization remain critical challenges of such a chemical proteomic platform. In this study, we designed and synthesized a diverse panel of twenty fragment-based probes containing natural product-based privileged structural motifs for small-molecule lead discovery. These probes were fully functionalized with orthogonal diazirine and alkyne moieties and used for protein crosslinking in live lung cancer cells, target enrichment via “click chemistry,” and subsequent target identification through label-free quantitative LC-MS/MS analysis. Pair-wise comparison with a blunted negative control probe and stringent prioritization via individual cross-comparisons against the entire panel identified glutathione S-transferase zeta 1 (GSTZ1) as a specific and unique target candidate. DepMap database query, RNA interference-based gene silencing and proteome-wide tyrosine reactivity profiling suggested that GSTZ1 cooperated with different oncogenic alterations by supporting survival signaling in refractory NSCLC cells. This finding may form the basis for developing novel GSTZ1 inhibitors to improve the therapeutic efficacy of oncogene-directed targeted drugs. In summary, we designed a novel fragment-based probe panel and developed a target prioritization scheme with improved stringency, which allows for the identification of unique target candidates, such as GSTZ1 in refractory lung cancer.

### Introduction

Displaying relatively simple structural complexity, small-molecule fragments can cover large chemical space and provide structurally diverse starting points for drug discovery¹. Fragment-based approaches have been successfully applied to discover new viable hits and diverse protein targets for developing targeted therapeutics, for instance, the discovery of the BCL2 inhibitor, navitoclax,²⁻⁴ and the FDA-approved BRAF inhibitor, vemurafenib⁵. These fragment hits are generally favorable for drug discovery, considering more hydrophilic character, higher ligand efficiency, and less sterically demanding groups⁶. Thus, fragment-based drug discovery is frequently used to prioritize well-validated targets (“target-based drug discovery”) and de-risk costly and time-intensive drug development efforts. However, per se this does not address the challenge that only a small proportion of the vast chemical space, which is available to engage biological targets, is currently being explored and utilized⁷. Recently, the Cravatt group has developed a powerful approach to tap into this larger chemical space by using smaller fragment-like chemical probes fully functionalized with photoreactive and bio-orthogonal reporter groups⁸. Fragment-like probes have distinct advantages over larger, more decorated drug-like molecules due to their smaller size and simpler structures, allowing them to engage target binding sites inaccessible to more developed molecules unless they were already optimized for interacting with that specific target. Thus, fragment-like molecules encode more target interaction flexibility and are therefore significantly better suited to probe uncharted biological target space.

Quantitative chemical proteomics is a powerful approach for identifying proteome-wide small molecule-protein interactions and novel druggable targets. Several chemical proteomics studies focusing on profiling fragment-protein interactions have demonstrated high efficiency and applicability of using fragments or fragment-like probes for new target and hit discovery⁸⁻¹⁰. However, it remains challenging to rationally design fragment-like probes and choose proper fragments for target discovery as these compounds should be drug-like and suitable for hit-to-lead follow-up optimization campaigns. Commonly utilized chemical scaffolds in drug discovery stem either from drug/lead-like molecules¹¹ or from privileged structural scaffolds^{8,12}. In our study, we built our panel of diverse fully functionalized probes based on a BioCore¹³, a privileged saturated and aromatic heterocyclic ring pair that represents a synergistic compromise of natural product elements into synthetic scaffolds, to proteome-wide fragment-based target identification. BioCores represent minimalist structural elements of bioactive natural products and lead/drug-like molecules, which are therefore well suited for identification of new targets; furthermore, they have high feasibility for subsequent structural modifications and serve as excellent starting points for drug discovery. However, as each fragment-like probe may be associated with multiple protein targets, target identification and prioritization are critical. Utilizing the unique properties of the entire fragment-like probe panel, we performed panel-wide cross-comparisons of target profiles to identify probe-specific targets. Through querying pharmacogenomics databases, such as DepMap¹⁴, target candidates can be further prioritized to enrich for phenotypically relevant targets.

Oncogenic alterations are common hallmarks of cancer and using small-molecule inhibitors to target these alterations is often effective in cancer management and treatment. In non-small cell lung cancer (NSCLC), specifically the lung adenocarcinoma (LUAD) subtype, KRAS^{G12C} is the most frequent KRAS mutation, but only 30-50% of patients harboring KRAS^{G12C} mutations respond to targeted drugs, such as the covalent KRAS^{G12C} inhibitor sotorasib^{15,16}. In NSCLC of the squamous cell lung cancer (LUSQ) subtype, KRAS mutations are rarely detected, but other oncogenic aberrations, such as FGFR1 amplification (˜20%) and DDR2 mutations (˜4%), have been described and are associated with cancer cell vulnerability towards drugs that target these tyrosine (Tyr) kinases. However, the efficacy of single agent infigratinib (a pan-FGFR inhibitor) and dasatinib (a multikinase DDR2 inhibitor) in clinical trials has been limited^{17,18}. Thus, there are currently no targeted therapies that are approved for LUSQ. In this study, to discover new druggable targets and chemical hits for treating drug-refractory lung cancers, we designed, synthesized, and applied a panel of fully functionalized photoactivatable fragment probes harboring BioCore structural elements. Probe target profiles were established by using mass spectrometry-based chemical proteomics and label-free quantitation. Through cross-comparison and pharmacogenomic database queries, we found that Glutathione S-transferase Zeta 1 (GSTZ1) is a unique target for one of the probes, and that its gene knockdown can enhance cancer cell response to targeting oncogenic drivers. Thus, the application of fully functionalized fragment probes with label-free quantitation MS analysis, combined with a stringent workflow for the parallel discovery of phenotypically relevant targets and their binding molecules may be useful for developing novel targeted therapies against drug-refractory diseases.

### Results

### Design and Synthesis of a Panel of Photoactivatable Fragment-Like Probes

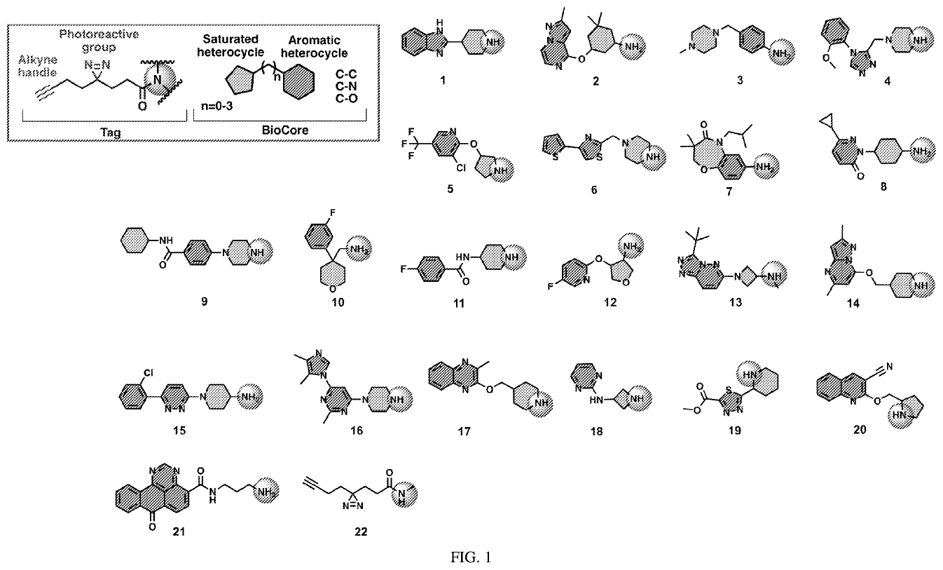

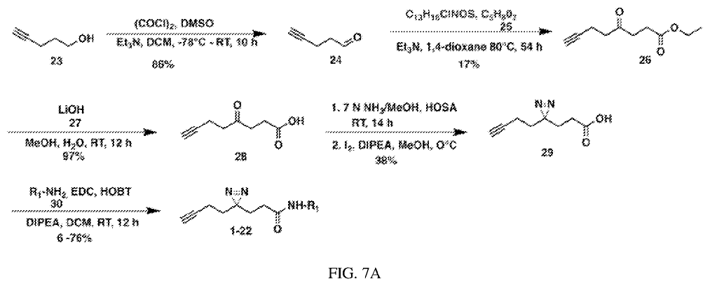

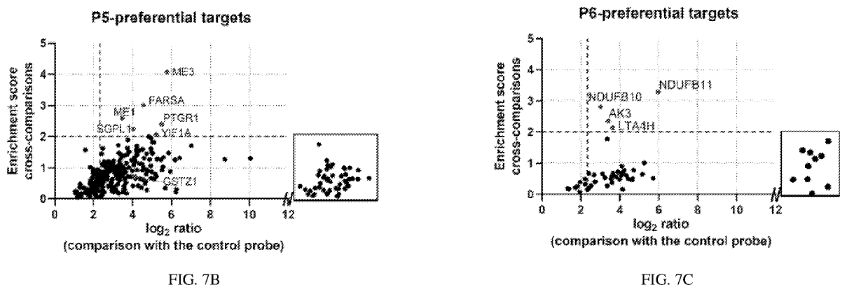

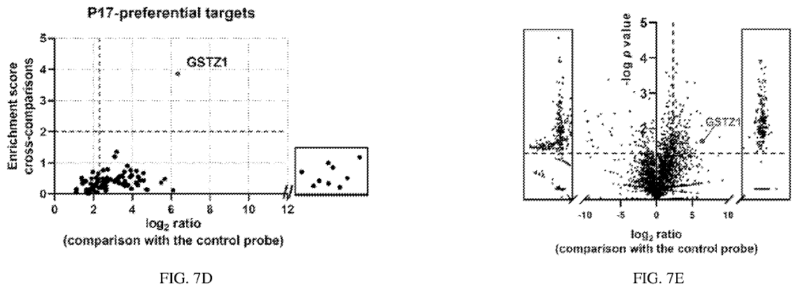

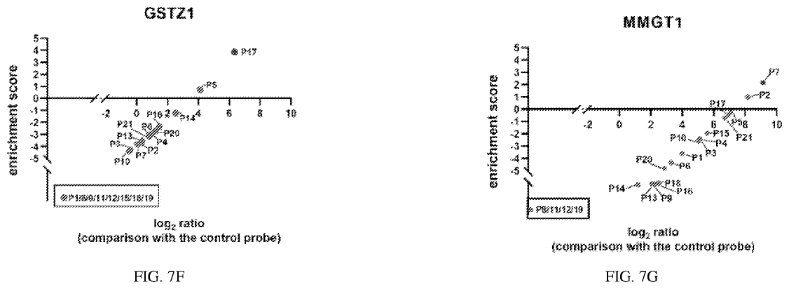

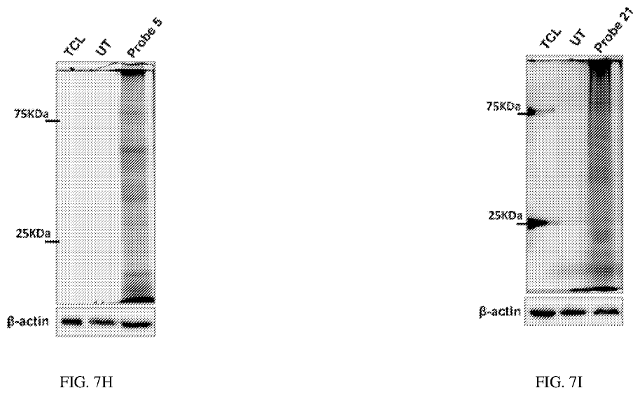

We constructed a panel of 20 photoactivatable fragment-like probes where the fragments represent a small yet diverse set of small molecules from a large library of HTS compounds. Fragments were prioritized based on desired physicochemical properties to improve the probability of synthetic tractability, drug-like properties, as well as the incorporation of BioCores (FIG. 1). These fragments were selected based on dissimilarity search among a larger (˜50K) commercially available diversity set of the Life Chemicals HTS compound collection. The distance matrix (MAXSUM, maximum sum of pairwise distances) was computed from molprint 2D fingerprints to generate a set of most diverse compounds (with the smallest sum of similarities to the other molecules) using Canvas interface (Schrodinger LLC)¹⁹. Furthermore, compounds were prioritized based on desired calculated physicochemical properties for leadlikeness (MW<350, cLogP<1.0-3.0, no reactive groups)²⁰ as well as incorporation of BioCores. BioCores are defined as a heteroaromatic ring connected by a linker (C—C, C—O, C—N) to a saturated heterocyclic molecule. As polycyclic aromatic rings are commonly incorporated as chemical motifs of DNA intercalating or DNA damage inducing reagents for developing anti-tumor drugs²¹, we also included such an additional scaffold for comparison (probe 21). All 21 commercial fragments harboring amine groups were conjugated to a linker with clickable alkyne and photoactivatable diazirine groups via simple amide coupling using the corresponding acid derivative. We used this probe set, and the methyl amide blunted probe 22 described by Parker and colleagues as a negative control⁸, for chemoproteomic experiments. The general procedure for the synthesis of the diazirine-alkyne handle is based on a previously reported sequence with minor modifications (FIG. 7A)^{8,22}. Taken together, we designed and synthesized 20 BioCore-containing photo-crosslinkers, one probe harboring a scaffold of DNA intercalating reagents, and one negative control probe suitable for proteome-wide target interrogation.

### Druggable Proteome Exploration in Live Lung Cancer Cells

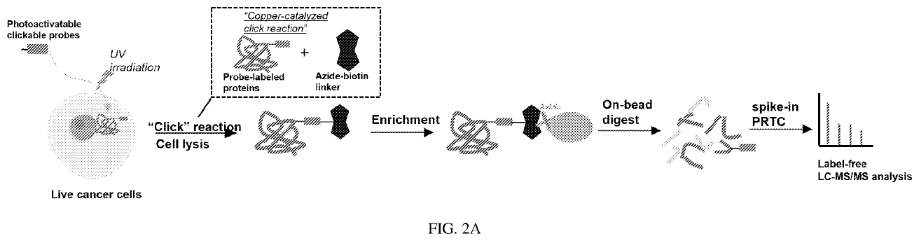

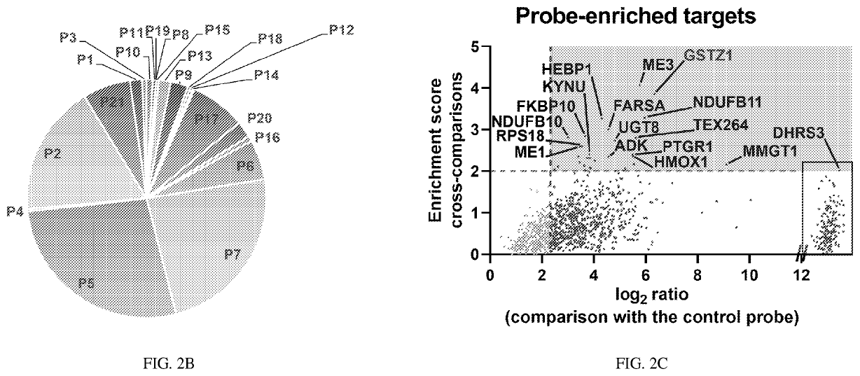

All probes were tested in live H1792 LUAD cells, which harbor a KRAS^{G12C} mutation, but are relatively resistant to KRAS^{G12C} inhibitors. Parallel batches of cells were treated with the indicated individual probes or DMSO, followed by exposure to UV light. Subsequently, cell lysates were incubated with an azide-biotin tag under copper-catalyzed click reaction conditions. Tagged proteomes were enriched by streptavidin beads and subjected to “on-bead” digestion with trypsin. Tryptic peptides were used for LC-MS/MS analysis and label-free quantitation (FIG. 2A). In total, more than 4,000 proteins were identified across all probes, and these proteins represented a wide range of the proteome, particularly mapping to the mitochondrion and the cytosol and being involved in protein translation and stability, as well as post-translational modifications. To quantitatively assess probe-enriched proteins, we performed a comparative analysis of proteins enriched by individual probes against other probes, including the negative control probe 22. Firstly, we compared each panel probe with the control probe 22, defining a fold change increase of protein intensity signals for any given probe of 5-fold (log₂(5)=2.32), as described previously by Parker and colleagues⁸. This cutoff yielded a significantly enriched protein set of 932 proteins. Notably, the number of probe-enriched target candidates varied greatly from probe to probe, with probes 2, 5, 6, 7 being the most promiscuous while some probes only enriched few (probe 18 and 19) proteins over probe 22, suggesting a probe-related labeling pattern (FIG. 2B). The enriched protein set also included 499 proteins that were not observed with the control probe, which nominally would make these proteins high priority targets. However, we noticed that almost all of these were broadly observed with multiple probes and thus had poor selectivity across the entire panel, which reduced confidence in representing specific interactions. We therefore also performed a panel-wide cross-comparison. To this end, in order to be considered selectively enriched, the protein signals of each biological replicate of a given probe of interest were required to be above the average of all other probes plus one standard deviation of the cross-panel comparison. The probe-versus-control probe and probe-versus-probe comparisons combined revealed that although a large number of potential target proteins were markedly enriched over the negative control probe and thus can be considered potential targets, most of these were similarly shared between several probes, which can reduce the confidence in these interactions (FIG. 2C). However, 31 proteins were furthermore enriched with greater selectivity by individual probes compared to the rest of the probe panel and were therefore considered higher confidence target candidates (FIG. 2C). These proteins mostly mapped to the mitochondria and endoplasmic reticulum and affected the electron transport chain. In addition, many targets exhibited interactions with several probes, and some of these displayed preferential interactions with one or a subset of probes. For instance, the prominent target candidates ME3, GSTZ1, and NDUFB11 were predominantly enriched by probes 5, 17, and 6, respectively. However, in addition to ME3, probe 5 also strongly enriched a group of other proteins such as FARSA, PTGR1, ME1, YIF1A, and SGPL1 (FIG. 7B). Probe 6 also enriched additional proteins, such as NDUFB10, AK3, and LTA4H (FIG. 7C). Interestingly, probe 17 enriched GSTZ1 much more than other proteins when compared to the probe panel (FIG. 7D), although it showed also many other significantly enriched putative targets when compared to the negative control probe alone (FIG. 7E), and, as it has not been optimized yet, should not be considered a selective probe for GSTZ1. However, probe 17 enriched GSTZ1 to much greater degree than any other probe in the panel suggesting a high-confidence interaction between probe 17 and GSTZ1 (FIG. 7F). In comparison, MMGT1 was also identified as a high-confidence target (FIG. 2C), but the difference between probe 7, which enriched MMGT1 to the largest extent over the control probe (p=0.015), and the other probes was not quite as pronounced as it was for probe 17/GSTZ1 (FIG. 7G). Thus, for the purpose of demonstrating the feasibility of the approach probe 17 and GSTZ1 were selected for the validation of probe-target specificity. In summary, we tested a total 22 fragment-like probes by using label-free quantitative proteomics and prioritized GSTZ1 as the most prominent target of probe 17 through intensive cross-comparisons with other probes.

Probe 17 Exhibits Specific Physical and Functional Interaction with GSTZ1

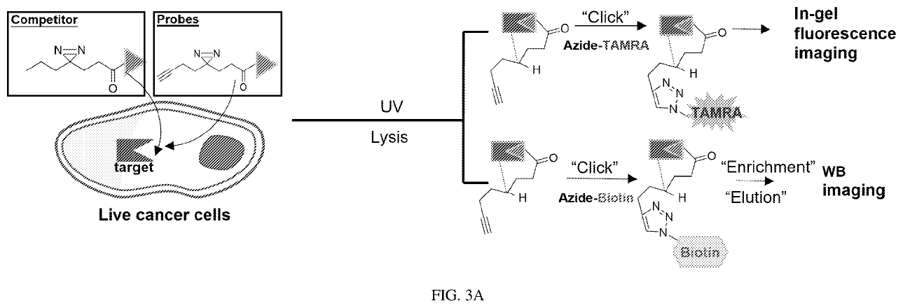

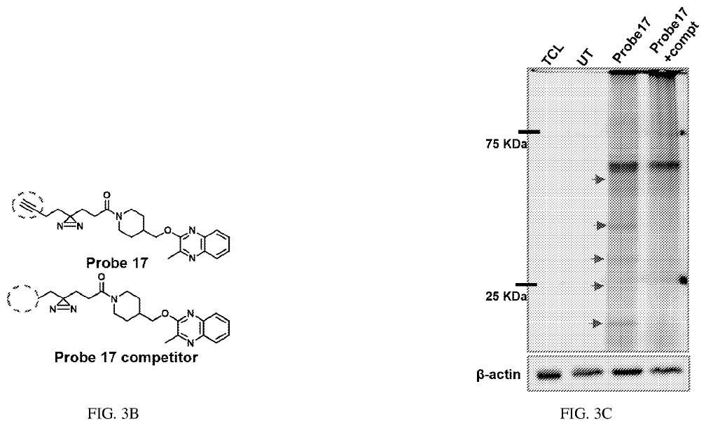

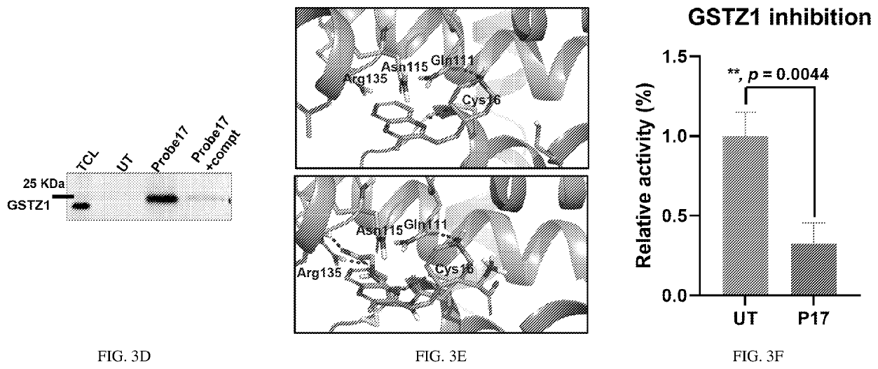

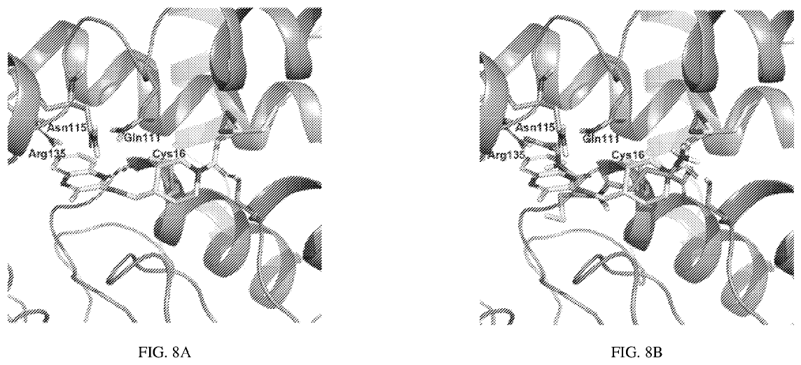

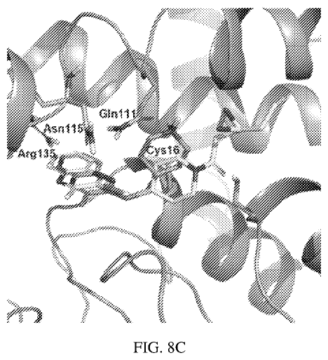

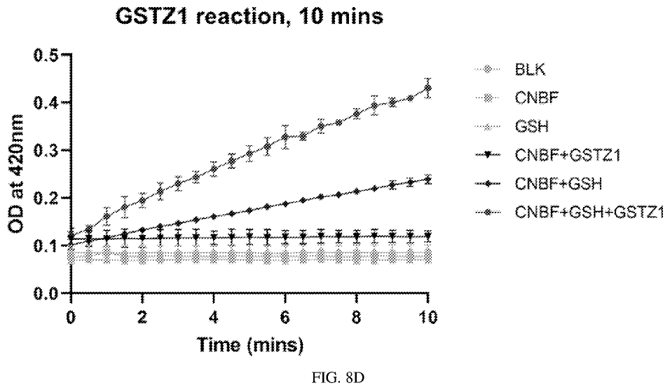

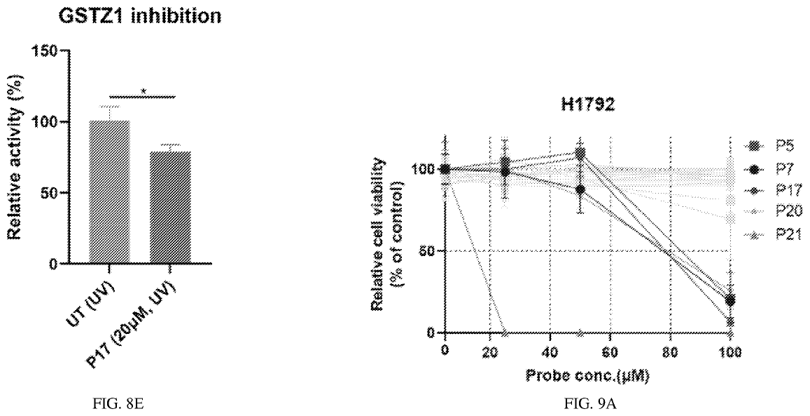

GSTZ1 belongs to the glutathione S-transferase (GST) family and displays glutathione (GSH)-conjugating activity and GSH-dependent isomerization activity, thereby participating in multiple biological processes, such as tyrosine metabolism and redox homeostasis²³⁻²⁵. GST family enzymes are attractive anti-cancer targets given their general cytoprotective roles in cancer cell survival and drug resistance²⁶, and targeting GSTs and impairing redox balance have emerged as effective strategies for developing novel anti-cancer therapeutics²⁷⁻³⁰. Proteome-wide probe 17 labeling and its interaction with GSTZ1 were validated through competitive imaging approaches, including in-gel fluorescence imaging and immunoblotting. UV-enabled crosslinking was conducted in live H1792 cells, and cell lysates were subjected to copper-catalyzed azide-alkyne cycloaddition as before, conjugating tagged target proteins with an azide-TAMRA fluorophore for imaging or an azide-biotin linker for target enrichment (FIG. 3A). To increase the power of validation confidence, we designed and synthesized a structurally similar competitor without the alkyne moiety of the reporter tag to compete for target engagement prior to treatment with probe 17 (FIG. 3B). Fluorescence imaging showed that compared to vehicle treatment, probes 5, 17, 21, efficiently labeled a wide range of proteins with different molecular weights and display differential proteome-labeling patterns between probes (FIG. 3C, FIGS. 7H and 7I). Furthermore, probe 17 labeling was blocked by the competitor, suggestive of a probe-specific labeling event (FIG. 3C). To assess if GSTZ1 is a target of probe 17, the probe 17-labeled proteome was tagged with biotin and extracted for affinity enrichment and detection by immunoblotting with a GSTZ1-specific antibody. As expected, GSTZ1 was specifically enriched by probe 17 and strongly competed by the competitor (FIG. 3D), indicating that probe 17 was physically bound to GSTZ1. To explore the potential binding pocket of probe 17 in GSTZ1, the BioCore of probe 17 was docked into the human GSTZ1 site by using Glide³¹ and the publicly available protein crystal structure of GSTZ1 (PDB: 1FW1) 32. The uppermost ranked binding pose based on docking and Glide scores of the fragment within the active site of GSTZ1 suggested the fragment occupies the active site in a similar orientation to GSH, and the key contacts are two hydrogen bond interactions between the quinoxaline and piperidine with the side chains of Cys16 and Gln111, respectively (FIG. 3E). In addition, not only the BioCore of probe 17, but also probe 17 similarly interacted with the GSTZ1 protein as GSH did (FIGS. 8A-8C). The docking complex shows that the fragment was predicted to occupy the GSH binding pocket of GSTZ1 with its binding pose resembling GSH. To investigate if probe 17 could directly inhibit GSTZ1 enzymatic activity, we carried out a GSTZ1 enzymatic activity assay and found that the GSTZ1 activity was reduced by 68% by probe 17 at 500 mM and upon UV crosslinking also at a low concentration of 20 mM (21% inhibition) (FIG. 3F and FIGS. 8D and 8E). These results suggest that probe 17 is a GSTZ1 inhibitor and that physical interaction may encompass occupying the putative GSH-binding pocket of GSTZ1.

GSTZ1 Expression is Associated with Decreased Patient Survival and Dependency of Specific Drug-Refractory NSCLC Cell Lines

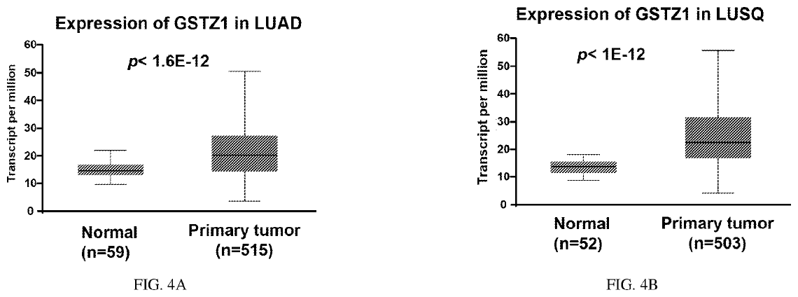

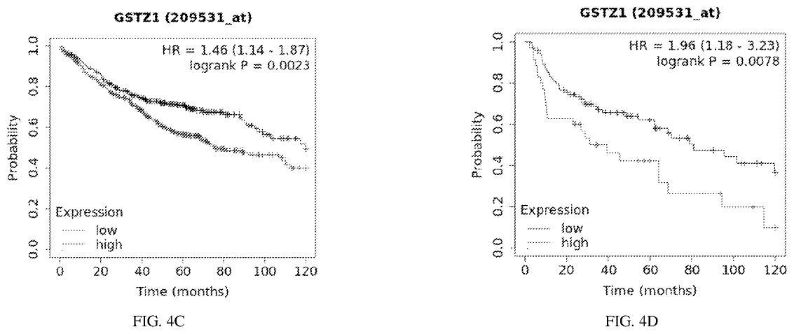

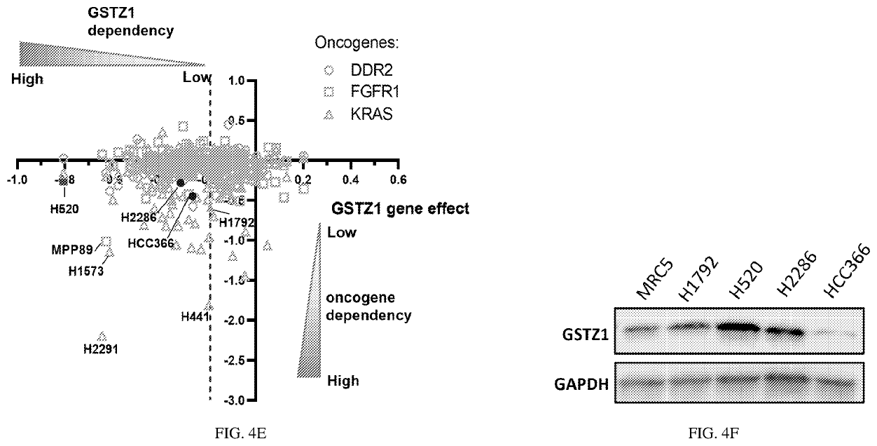

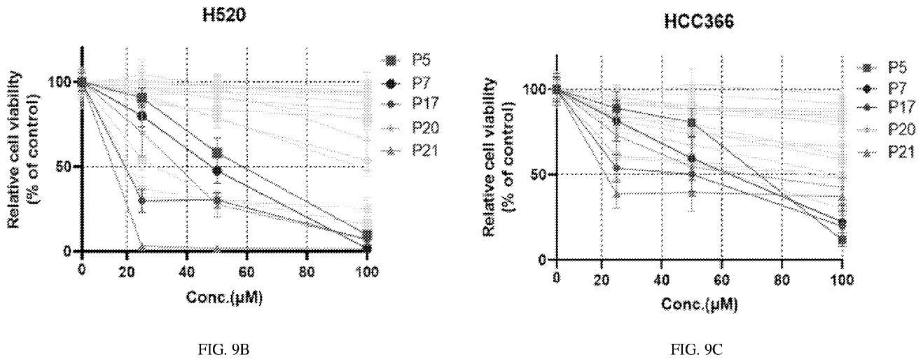

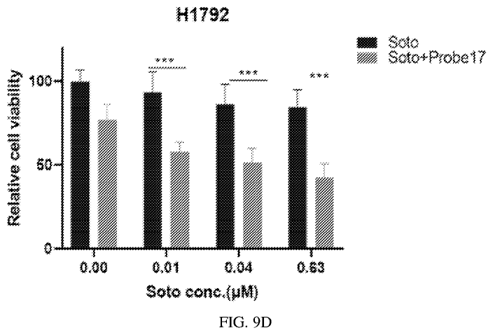

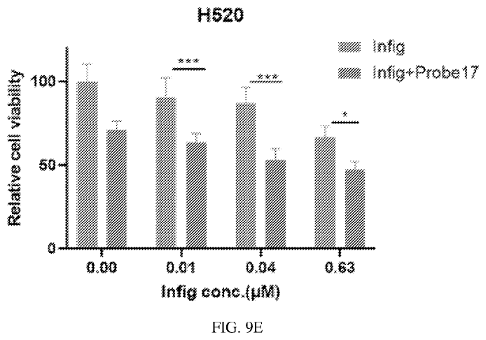

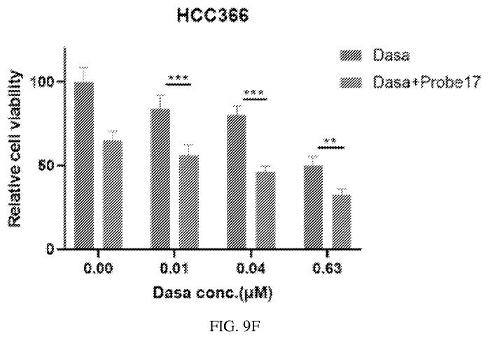

To assess the GSTZ1 expression status in lung cancer patients, we queried the GSTZ1 gene in the publicly accessible TCGA³³ and GEO³⁴ genomic databases. GSTZ1 was found to be significantly upregulated in both LUAD and LUSQ tumors vs. normal lung tissues (FIGS. 4A-4B). Kaplan-Meyer survival curves comparing high and low expression of GSTZ1 in LUAD and LUSQ patient groups showed that high GSTZ1 expression was significantly correlated with poor prognosis of LUAD (FIG. 4C) and LUSQ patients (FIG. 4D), suggesting therapeutic potential of GSTZ1 as a druggable target for treating NSCLC. To evaluate GSTZ1 gene dependency in drug-refractory NSCLC cell lines that represent medical challenges for targeted therapy, we queried both GSTZ1 and oncogenic driver genes, such as KRAS (mostly LUAD), FGFR1 (LUSQ), and DDR2 (LUSQ), in the DepMap pharmacogenomic database¹⁴. The DEMTER2 score generated from combined siRNA datasets (Achilles+DRIVE+Marcotte) was used to plot and represent gene dependency with lower scores indicating higher dependency. This analysis suggested varying GSTZ1 gene essentiality across different types of lung cancer cell lines (FIG. 4E). However, we noted that several lung cancer cell lines that harbor oncogenic drivers, but only partially respond to the corresponding targeted drugs, showed pronounced GSTZ1 dependency. This panel included the LUAD cell line, H1792, which was used as a positive control since it poorly responded to sotorasib or ARS1620 despite the presence of a KRAS^{G12C} mutation (FIGS. 9A-9F). LUSQ cell lines, including H520 (FGFR1 overexpression), H2286 (DDR2^{1638F}), and HCC366 (DDR2^{L239R}), were selected because they only partially respond to targeted drugs, such as the FGFR inhibitor infigratinib and the SRC/DDR2 inhibitor dasatinib, respectively, and represent molecularly defined lung cancer subtypes for which no targeted therapies are currently approved. Furthermore, DepMap analysis suggested that GSTZ1 was relatively more essential in these cells than in H1792 cells (bottom left quadrant, FIG. 4E). With the exception of HCC366 cells, H1792, H520, and H2286 cells were also found to have higher GSTZ1 protein levels compared with non-cancerous MRC5 lung fibroblasts (FIG. 4F), suggesting a targetable potential for lung cancer treatment. Together, these results indicate that GSTZ1 is not a generally essential gene across all types of lung cancer cells, but that some LUAD and LUSQ tumors and drug-refractory cell lines may exhibit co-dependency of GSTZ1 and specific oncogenes.

### GSTZ1 Cooperatively Supports Drug-Refractory Cancer Cell Survival

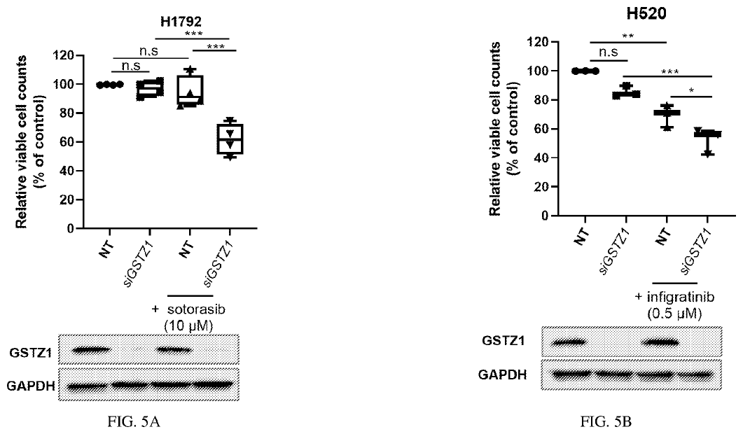

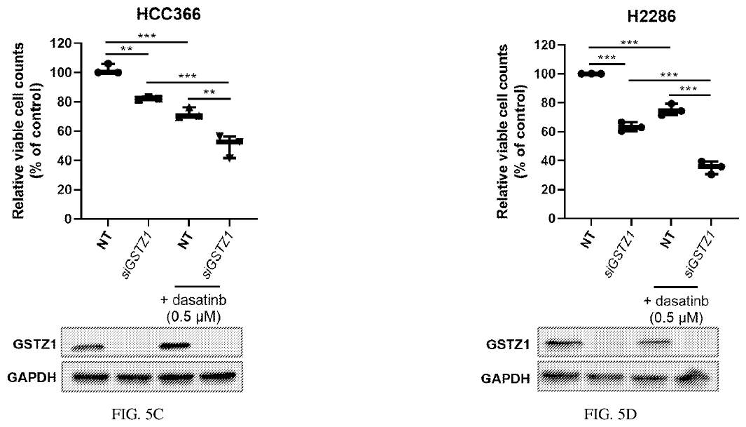

To evaluate the effect of GSTZ1 on the viability of drug-refractory lung cancer cells, especially in the context of oncogenic inhibition, we silenced the expression of GSTZ1 in H1792, H520, H2286, and HCC366 cells using siRNAs in the presence or absence of the respective targeted drugs, e.g. sotorasib in H1792 cells, infigratinib in H520 cells and dasatinib in HCC366 and H2286 cells. In all four cell lines, GSTZ1 was efficiently knocked down, which had the most pronounced effects on viability of DDR2-mutant cells (FIGS. 5A-5D). In contrast, H1792 cells were not affected by silencing of GSTZ1, which was in line with the GSTZ1 dependency scores from the DepMap RNAi database (FIG. 4E). Importantly, GSTZ1 knockdown significantly enhanced the cell response to these clinically relevant drugs in all four cell lines (FIGS. 5A-5D), which was consistent with the cell sensitization effect imposed by probe 17 (FIGS. 9D-F). Probes 17, 20, 7, 5, and 21 displayed activity toward multiple tested cell lines, with probes 17 and 21 being more potent (FIGS. 9A-C). Probe 21 was highly active in H1792 and H520 cells, which raises concerns about its promiscuous target profile, with 5 candidate target proteins being identified for probe 21 in addition to an increased DNA intercalating potential (FIG. 2B). Considering a general lack of cellular effects observed for most tested fragment probes, probe 17 displayed moderate single agent activity, and also significantly enhanced the sensitivity of these lung cancer cells towards the relevant oncogene-targeting drugs sotorasib, infigratinib, and dasatinib (FIGS. 9D-F). This was consistent with sensitivity of these cells towards genetic knockdown of GSTZ1. Notably, as it has not been optimized yet, probe 17 is not a selective probe molecule for GSTZ1 and features multiple putative targets (FIG. 9E), some of which are likely to contribute to the cellular effects. However, as UV-crosslinking and 20 mM of probe 17 at least partially inhibited GSTZ1 (FIG. 8E), it is likely that GSTZ1 inhibition is one of the factors that contribute to the overall cellular effects of probe 17. These results indicated that the panel of targets hit by probe 17 alone are only moderately relevant for cell survival yet modulate activity of oncogene-directed targeted drugs, which considering that GSTZ1 was a prominent target of probe 17 supported the observations from GSTZ1 gene silencing. In summary, targeting GSTZ1 conferred moderate sensitivity to NSCLC cell lines, but prominently enhanced efficacy of oncogene-directed targeted drugs, suggesting that GSTZ1 cooperates with these oncogenic alterations in supporting cancer cell survival.

### GSTZ1 Modulates Tyr Phosphorylation of Oncogenic Drivers

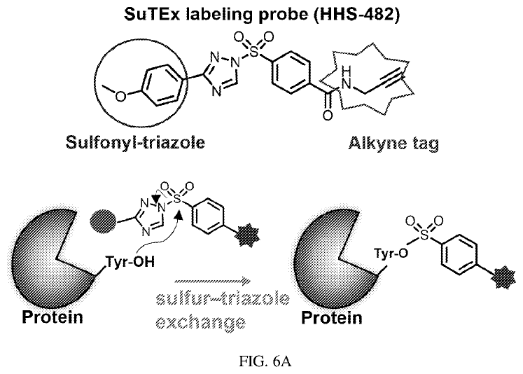

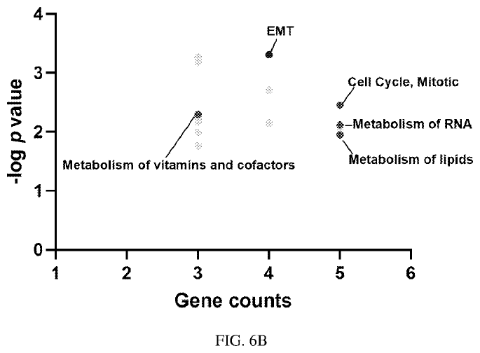

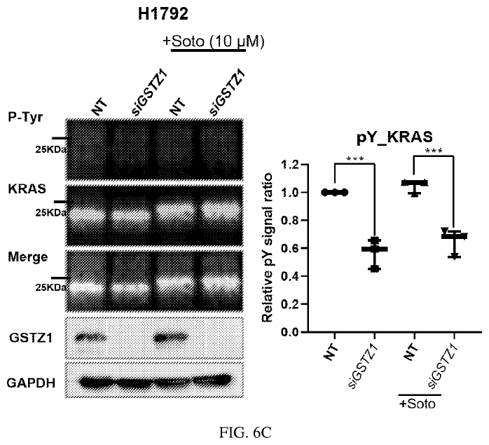

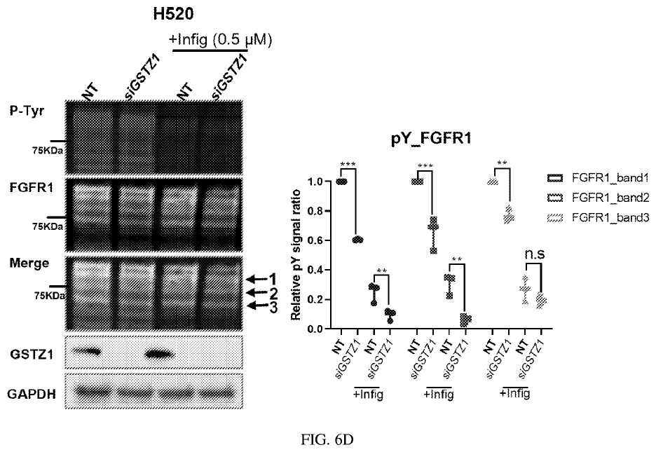

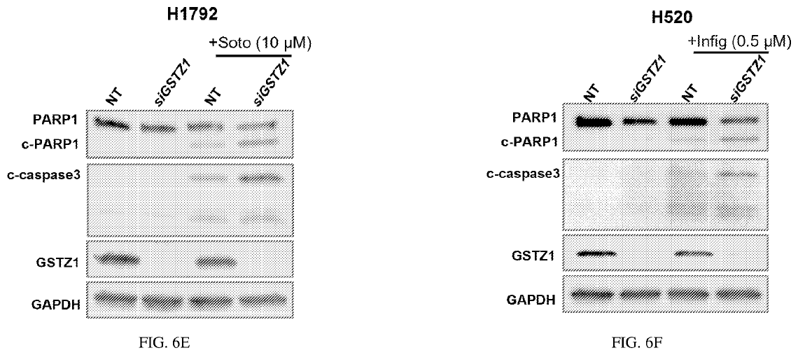

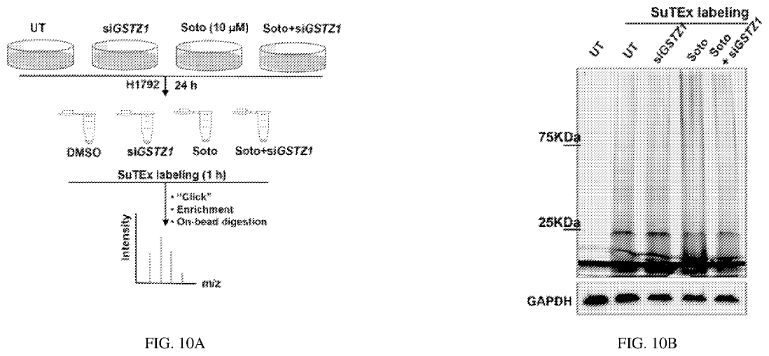

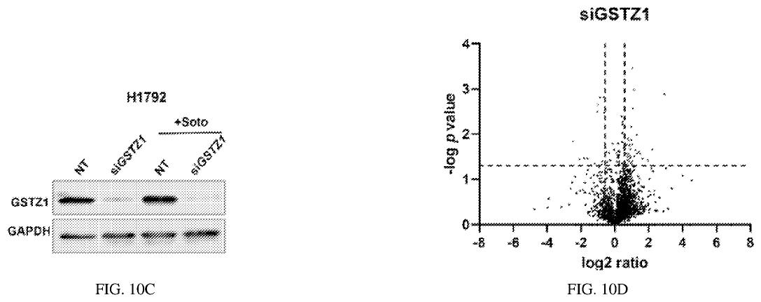

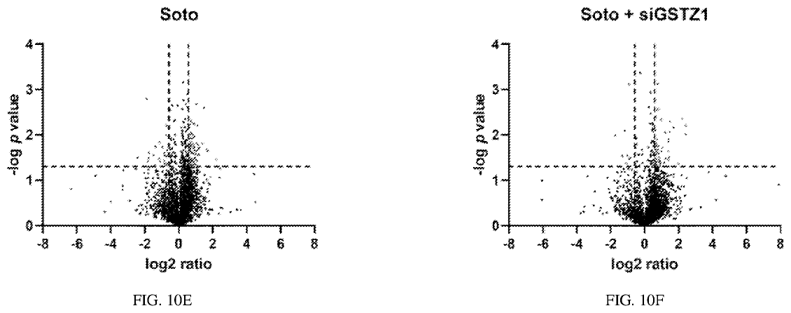

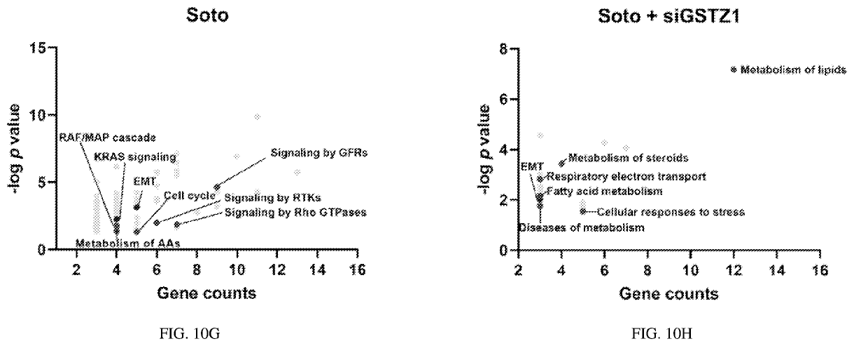

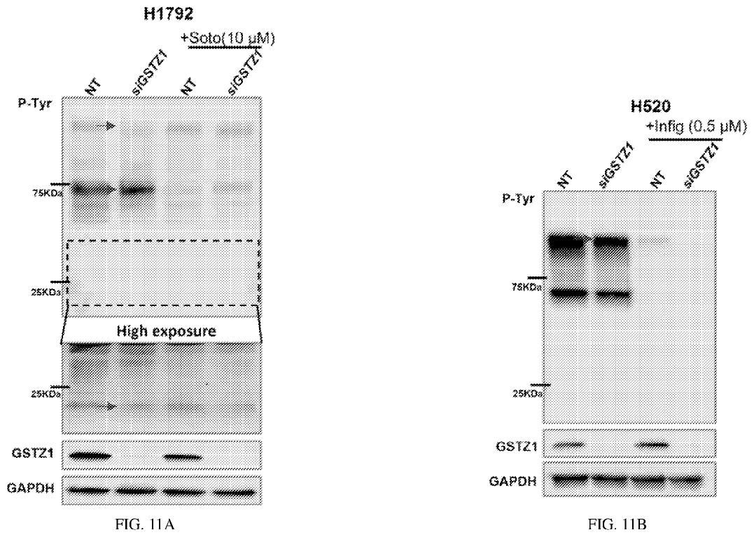

GSTZ1 is the penultimate enzyme in the tyrosine-degradation pathway³⁵ and modulates tyrosine-related signaling³⁶. Thus, we hypothesized that GSTZ1 activity affects tyrosine biology in lung cancer cells. Biochemical alterations in hotspots and signaling networks often mirror changes in reactivity of specific protein residues and several chemoselective probes have been developed to profile protein residue reactivity and discover diverse hotspot targets on a proteome-wide scale³⁷⁻⁴⁰. We therefore employed Tyr-reactive sulfur-triazole exchange (SuTEx) probe labeling, which has been used previously as a readout of alteration in protein Tyr reactivity in living cells³⁹. Proteome-wide Tyr residue reactivity profiling was enabled through sulfur-triazole exchange chemistry (FIG. 6A), and the labeled proteome was processed by click chemistry-based linker conjugation, enrichment, on-bead digestion, and label-free LC-MS/MS analysis (FIG. 10A). Gel-based imaging using H1792 cells and comparing vehicle vs. sotorasib and/or GSTZ1 knockdown showed that sotorasib treatment globally modulated protein Tyr residue reactivity profiles and that this was modulated by GSTZ1 silencing (FIGS. 10B and 10C). Subsequent LC-MS/MS analysis revealed 55 proteins, 106 proteins, and 66 proteins that were significantly altered upon silencing GSTZ1, sotorasib treatment or the combination of both, respectively (FIGS. 10D-10F). To understand biological processes and signaling pathways associated with these altered proteins, we performed a pathway analysis, which showed that sotorasib treatment enriched several signaling pathways known to be associated with RAS signaling and resistance to KRAS^{G12C} inhibitors, such as epithelial-mesenchymal transition (EMT), KRAS signaling, growth factor receptor^{41,42}, the cell cycle⁴³, amino acid metabolism⁴⁴ and Rho GTPase-mediated signaling⁴⁵ (FIG. 10G). Notably, GSTZ1 silencing also enriched EMT, cell cycle, and metabolism pathways (FIG. 6B), and EMT pathway modulation was retained upon combined sotorasib treatment and GSTZ1 silencing (FIG. 10H), suggesting GSTZ1 may be involved in cell sensitivity to sotorasib by modulating EMT, which in H1792 cells has been shown to depend on FGFR1 signaling⁴¹. In light of these findings and that GSTZ1 was also known to degrade tyrosine and alter tyrosine kinase and growth factor receptor activity (e.g., IGFIR Y1161)³⁶, we next assessed the effect of GSTZ1 silencing on protein Tyr phosphorylation in both KRAS-mutant H1792 and H520 cells, the latter of which feature gene amplification of the growth factor receptor FGFR1. As expected, GSTZ1 knockdown reduced protein tyrosine phosphorylation of several proteins, some of which maintained low levels of protein Tyr phosphorylation upon treatment with sotorasib (FIG. 11A) or infigratinib (FIG. 11B). Interestingly, two-color immunoblotting for phosphotyrosine and KRAS or FGFR1 in H1792 and H520 cells, respectively, suggested that GSTZ1 knockdown directly attenuated the Tyr phosphorylation of KRAS (FIG. 6C) and FGFR1 (FIG. 6D). Considering that these two oncoproteins impact cell survival, we next determined the effects of GSTZ1 silencing on cell apoptosis. Notably, enhanced caspase 3 and PARP1 cleavage indicated that in both H1792 and H520 cells combined GSTZ1, and oncogene-targeted inhibition induced more cell apoptosis than single drug treatment alone (FIGS. 6E-6F). In conclusion, these results suggest that protein Tyr residue reactivity alterations can reflect compensatory resistance mechanisms in response to drug treatment, and that targeting of GSTZ1 could potentially disrupt these compensatory signals by modulating the Tyr phosphorylation of oncoproteins such as KRAS^{G12C} and FGFR1.

### Discussion

Targeted drugs have provided significant benefits for NSCLC patients with oncogenic alterations, such as KRAS^{G12C} mutation. However, the clinical efficacy of targeted drugs is limited due to a plethora of resistance mechanisms. Therefore, to improve the efficacy of these clinical drugs, the discovery of novel therapeutic targets and development of the matched chemical matter to drug those targets is urgently needed. In this study, we designed and synthesized a panel of structurally diverse fragment-based probes for identifying novel ligandable targets in drug-refractory NSCLC cells. By using label-free chemical proteomics, we identified GSTZ1 as a prominent new target protein involved in modulating the cellular response to oncogene-targeted drug treatment potentially through modulation of biologic reactivity and phosphorylation of protein Tyr residues, including those on the respective oncoproteins.

Structurally diverse BioCore-containing probes have great potential for addressing some challenges noted in traditional target- and fragment-based screens (e.g., not in live cells, low affinity, hard for target validation)⁴⁶. To explore the ligandable proteome in drug-refractory cells, we conducted target profiling with these probes by using a label-free chemical proteomics platform, which is in contrast to previously published FBDD studies that use labeling methods such as SILAC⁸ and TMT¹⁰. Compared to latter labeling methods, we constructed a simplified sample preparation workflow and processed a greater number of MS samples without being limited by the number of isobaric tags or metabolic labels. In addition, isobaric mass tagging approaches overemphasize detection of common hits and may miss unique proteins that would only bind to a specific probe. Furthermore, chemical labeling often suffers from ratio compression and may underestimate panel-wide differences for proteins uniquely enriched by specific probes. Moreover, label-free quantification can be superior for protein quantification and proteome coverage in the context of using cell line samples that have significant biological differences^{47,48}. To facilitate target identification and prioritization, we developed an analytical platform by incorporating pharmacogenomic database searching (DepMap) and stringent statistical cross-comparisons between probes. DepMap querying effectively filtered out commonly essential proteins and identified phenotype-relevant targets. Cross-comparisons of probe target profiles quantified and ranked specificity of proteins towards their probes. Therefore, we found that such two-dimensional analyses efficiently prioritized GSTZ1 as a unique target of probe 17 for further validation.

The further utility of targeting GSTZ1 was examined in selected lung cancer models, including KRAS^{G12C}., FGFR1- and DDR2-driven lung cancer cells. Therapeutic resistance to KRAS^{G12C} inhibitors has been noted in preclinical tumor models and clinical trials. Many studies have shown intrinsic resistance to the inhibition of KRAS activity and downstream RAS signaling^{15,42}. However, the mechanisms leading to global changes in the tyrosine phosphoproteome are incompletely understood. In addition, proposed drug combinations are usually limited to involving known targets. In LUSQ patients, there is no conclusive evidence supporting the clinical use of DDR2- or FGFR1-targeted drugs. For instance, a preclinical study found that DDR2-mutated LUSQ cells respond poorly to selective DDR2 inhibition¹⁷, and clinical trials reported only partial responses to FGFR1 inhibitors in LUSQ patients despite stratification by FGFR1 amplification^{49,50}. In our study, we tested a panel of diverse fragment-like chemical probes for NSCLC cell viability and profiled their cellular targets using chemical proteomics. GSTZ1 was identified as a prominent target involved in cell survival. GSTZ1 is a multifunctional enzyme important for the detoxification of electrophilic molecules by conjugation with glutathione, tyrosine metabolism^{23,35}, redox homeostasis²⁴, and resistance to the multi-kinase inhibitor sorafenib in hepatocellular carcinoma²⁵. In lung cancer, GSTZ1 has been reported to be upregulated in many subtypes of lung cancer²³. However, the biological relationship of GSTZ1 with specific oncogenic alterations in lung cancer has not been established, and there are no known potent or selective GSTZ1 inhibitors for therapeutic evaluation. To understand the biological role of GSTZ1 in lung cancer cells with oncogenic alterations, we applied proteome-wide Tyr residue reactivity profiling enabled by a chemoselective SuTEx probe³⁷⁻⁴⁰. Protein residue-reactive probes have been explored as powerful tools to target a wide range of diverse proteins regardless of their tractability, and also biochemical alterations of proteins resulting from mutations, protein-protein interactions, and PTMs³⁷⁻⁴⁰. However, this approach has been mainly pursued with Cys- and Lys-reactive probes, and Tyr-reactive probes have not been explored in characterizing molecular or pathway alterations in response to drug treatment. In our study, we found that, exemplified by using H1792 cells, protein Tyr reactivity profiles could capture critical signaling pathways known to mediate drug resistance and cell survival such as growth factor receptor signaling and amino acid metabolism. In line with this finding, the loss-of-function study of GSTZ1 was found to globally disrupt protein Tyr reactivity by decreasing Tyr phosphorylation of FGFR1 in H520 cells, which is biologically meaningful as attenuating the Tyr phosphorylation of FGFR1 was reported to enhance the activity of KRAS^{G12C} inhibitors in H1792 cells⁴¹. Consistently, GSTZ1 knockdown reduced the Tyr phosphorylation of KRAS in H1792 cells and increased cell response to KRAS^{G12C} inhibition, potentially by modulating its enzymatic activity and signaling. In addition to oncogenic driver modulation, it could be interesting to evaluate, if GSTZ1 can modulate the activity of some hotspot proteins downstream of these oncogenic alterations such as AKT (e.g., Y176) and SHP2 (e.g., Y542, Y580) as they are broadly involved in cancer cell survival and drug resistance⁵¹⁻⁵⁵. The underlying mechanisms by which GSTZ1 modulates tyrosine phosphorylation of these oncoproteins warrant further investigation. Moreover, the identified BioCore 17 could be a candidate chemical scaffold for developing either single-targeted GSTZ1 inhibitors or multi-targeted drug-like molecules^{56,57}. These conceptual insights and technical advances are expected to provide the rationale for using GSTZ1 inhibitors for combination therapies against drug-refractory NSCLCs, particularly KRAS^{G12C} LUAD and LUSQ, thereby opening new horizons for employing efficacious combined therapies to treat drug-refractory tumors.

In conclusion, we developed an advanced chemical biology workflow to simultaneously discover novel, potentially therapeutically relevant targets and their small-molecule modulators in drug-refractory lung cancer cells. This workflow includes a panel of fully functionalized fragment-based probes, chemical proteomics, improved data analysis and target prioritization, and cellular validation, which could allow us to explore the druggable target space in intact biological systems (e.g., live cancer cells) and understand target protein mechanisms for cancer cell vulnerability and drug resistance.

### Methods

### Cell Culture and Reagents

H1792, H520, H2286, HCC366 were provided by the Moffitt Lung Cancer Center of Excellence cell line core. All cell lines have been authenticated by STR analysis and tested negative for mycoplasma. Cells were cultured in RPMI1640 media supplemented with 10% FBS and maintained in a humidified atmosphere containing 5% CO₂ at 37° C. Reagents include: sotorasib (Chemietek), infigratinib (BGJ398, Chemietek), dasatinib (LC LABORATORIES), Azide-PEG3-Biotin (Sigma), TAMRA-Azide-Biotin (Kerafast), copper sulfate (Fisher), Tris(2-carboxyethyl) phosphine hydrochloride (TCEP, Sigma), Tris [(1-benzyl-1H-1,2,3-triazol-4-yl)methyl]amine (TBTA, Sigma), 4-Chloro-7-nitrobenzofurazan (CNBF, Sigma), recombinant GSTZ1 proteins (Sino Biological), L-Glutathione (Sigma), Isopropyl β-D-1-thiogalactopyranoside (IPTG, American Bio), Dithiothreitol (DTT, Fisher), Imidazole (Acros Organics), Triton X-100 (Fisher), TEV protease (produced in-house from Addgene plasmid #882). All fragment-based probes were synthesized in house and dissolved in DMSO as 50 mM stock solutions and stored at −20° C. until use.

### Cell Viability Assay and Synergy Calculations

Cells were seeded in Corning 384-well black walled/clear bottom microplates with a density of 1,000 cells/well and treated with indicated drugs and concentrations following cell attachment for 24 h. After 72 h or 96 h, as indicated, cell viability was evaluated with CellTiter-Glo (Promega, Madison, WI) according to manufacturer's recommendations. An M5 Spectramax plate reader (Molecular devices) was used to read the plates with 500 ms integration. Data was processed and analyzed using Excel and GraphPad Prism 9.0.

### RNA Interference

siRNAs included ON-TARGETplus SMART pools and individual siRNAs. SMART pool GSTZ1 (Horizon, L-011290-00-0005) and ON TARGET plus non-targeting (Horizon, D-001810-10-20) were purchased from Dharmacon. siRNAs were resuspended in 1×siRNA buffer diluted with RNase-free water, aliquoted, and stored at −80° C. siRNA stocks were thawed on ice, diluted with Opti-MEM™ (31985062, ThermoFisher, MA), and mixed well with Lipofectamine™ RNAiMAX (13778150, ThermoFisher, MA). Transfection of siRNAs was performed in 6-well plates following the manufacturer's protocol. Knockdown efficiency was monitored by Western blotting. Cells were plated with a density of 3×10⁵ cells/well and treated with siRNAs or drugs for the indicated time. Cells were trypsinized, harvested, washed, and resuspended in 1 mL RPMI1640 culture media. 20 μL of cell suspension was transferred in triplicate to Eppendorf tubes, followed by trypan blue staining and counting of viable cells. The leftover cells were collected and subjected to immunoblotting analysis. Data were analyzed using Image Studio Lite Ver 5.2 and GraphPad Prism 9.0.

### Immunoblotting

Cells were harvested and washed with PBS and lysed in RIPA buffer (150 mM NaCl, 50 mM Tris-HCl, pH 7.4, 5 mM EDTA, 1% NP40, 0.1% SDS) containing complete protease inhibitor cocktail (Roche, 11873580001) and phosphatase inhibitor cocktail 2 (Sigma, P5726) for 30 min on ice. The Coomassie Plus (Bradford) protein assay (23236, ThermoFisher, MA) was conducted for measuring concentrations of resulting supernatants. Samples were prepared by adding 4× Laemmli sample buffer and heat-denaturing for 5 minutes. All samples or total cell lysates were resolved by SDS-PAGE and then transferred onto PVDF membranes. 5% non-fat milk was utilized for blocking of non-specific binding. Blots were incubated with primary antibodies overnight at 4° C. For immunoblotting, primary antibodies were purchased from Cell Signaling: PARP1 (#9542), cleaved caspase-3 (#9661), Phospho-tyrosine Mouse mAb (P-Tyr-100, #9411), FGFR1 (#9740). GAPDH (#60004-1-Ig) and GSTZ1 (#14889-1-AP) were purchased from Proteintech. Secondary antibodies were either from LI-COR: IRDye® 680RD Goat anti-Mouse IgG (#926-68070), IRDye® 800CW Goat anti-Rabbit IgG (#926-32211) or from GE Healthcare: HRP-conjugated anti-rabbit (NA934) or anti-mouse (NA931) were applied and incubated with PVDF membranes for 1 h. Images were obtained using the Odyssey Fc Imaging system (LI-COR, Lincoln, NE). Densitometry was analyzed using Image Studio Lite Ver 5.2.

### Photocrosslinking in Cancer Cells

Cells were grown in 10 cm dishes to ˜90% confluence at the time of treatment. Cells were carefully washed twice with Dulbecco's phosphate buffered saline (DPBS) and replenished with fresh serum-free RPMI 1640 media containing indicated probes (20 mM), competitors, or DMSO. Without competitors, cells were incubated with probes for 30 min at 37° C. and then exposed to 365 nm UV light for 15 min on ice. With competitors, cells were pre-treated with competitors for 30 min and photoactivated for 15 min. Probes were then added to cells for an additional 30 min incubation. For no UV experiments, cells were incubated for 15 min under ambient light. Cells were harvested in cold DPBS and pellets were washed with cold DPBS (2 X). Pellets were either directly processed or kept frozen at −80° C. until use. Cells pellets were lysed for 30 min in cold DPBS (100-300 uL) per culture dish supplemented with 1% NP-40, followed by sonication (5 pulses, 4 s on, 20 s off, output setting=4). Lysates were centrifuged at 20,817×g for 20 min, and supernatant (soluble proteome) was used for further processing. Protein concentrations were determined using Bradford assays.

### Gel-Based Detection of Labeled Proteins

To 1 mg of protein, were added a mixture of TBTA (Tris((1-benzyl-4-triazolyl)methyl)amine, 60 μL/sample, 1.7 mM in 1:4 DMSO: t-BuOH stock), CuSO₄ (20 μL/sample, 50 mM in H₂O), TCEP (tris(2-carboxyethyl) phosphine, 20 μL/sample, 50 mM in DPBS, made fresh) and Biotin-Azide conjugate (10 μL/sample, 10 mM in DMSO stock). Each sample was rotated at room temperature for 2 h. To each resultant a cold 4:1 mixture (600 μL) of methanol (MeOH)/chloroform (CHCl₃) was added followed by cold PBS (100 μL) on ice. The resulting cloudy mixture was centrifuged (5,000×g, 10 min, 4° C.) to fractionate the protein interface from the organic and aqueous solvent layers. A MeOH:CHCl₃ (1:1, 1 mL, 2×) mixture was used to wash protein discs thoroughly. Protein discs were sonicated in cold 4:1 MeOH:CHCl₃ (2 mL) mixture. The remaining precipitate was pelleted by centrifugation (5,000×g, 10 min, 4° C.). The pellet was transferred to 5 mL microcentrifuge tubes and then resuspended in a freshly-prepared solution of proteomics-grade urea (320 μL, 6 M Urea in DPBS,) containing 80 μL of 10% SDS and then dissolved by sonication. To each solution, 4 mL DPBS was added (final conc: 0.2%, total volume) and incubated with pre-equilibrated streptavidin agarose (100 μL 1:1 slurry, Pierce) for 2 hrs at ambient temperature on a rotator. The streptavidin beads were collected by centrifugation (1,400×g, 2 min) and sequentially washed with 0.2% SDS in DPBS (2×1 mL), detergent-free DPBS (3×1 mL), and H₂O (3×1 mL) to remove unbound protein, excess detergent, and small molecules. Aliquots (30 μl) of 4× Laemmli buffer were added to samples and heated to 95° C. with beads and then incubated for 30 min. Eluates were extracted and used for Western Blotting. Aliquots (50 ug) of protein were prepared and treated with TBTA (3 μL/sample, 1.7 mM in 1:4 DMSO: t-BuOH stock), CuSO₄ (1 μL/sample, 50 mM), TCEP (1 μL/sample, 50 mM) and TAMRA-Azide conjugate (0.2 μL/sample, 10 mM). After 2 h incubation, 4× Laemmli buffer was added and samples were resolved using SDS-PAGE gels. Gels were subjected to fluorescence imaging or Western Blotting.

### Ms-Based Analysis of Crosslinked Proteins

Protein pellets were transferred to 5 mL microcentrifuge tubes and then resuspended in a freshly-prepared solution of proteomics-grade urea (320 μL, 6 M Urea in DPBS) containing 80 μL of 10% SDS and dissolved by sonication. To each solution, 4 mL DPBS was added (final conc: 0.2%, total volume) and incubated with pre-equilibrated NeutrAvidin agarose resin (100 μL 1:1 slurry, Pierce, Cat. 29201) for 2 hrs at ambient temperature on a rotator. The NeutrAvidin beads were collected by centrifugation (1,400×g, 2 min) and sequentially washed with 0.2% SDS in DPBS (2×2 mL), detergent-free DPBS (2×2 mL), and 50 mM ammonium bicarbonate (AMBIC, 2×2 mL). Beads were resuspended with 200 μL 50 mM AMBIC and incubated with TCEP 50 μL (100 mM in 50 mM AMBIC) for 30 min at 37° C. Iodoacetamide (50 μL of 200 mM in 50 mM AMBIC) was added and incubated for 30 min at ambient temperature. The beads were again washed with 2×1 mL 50 mM AMBIC and resuspended in 100 μL of fresh 50 mM AMBIC and treated with 2 μg of sequencing grade modified trypsin (Promega, #V5111) at 37° C. overnight. These samples were acidified with formic acid (final conc: 5%) and centrifuged (1,000×g, 2 min). The supernatant was collected and desalted through ZIPTIPs, and the desalted peptide mixture were dried in vacuum and reconstituted with HPLC buffer (2% acetonitrile, 0.1% formic acid) for LC-MS/MS analysis. Tryptic peptides of avidin were excluded from MS2 analysis to maximize detection of the protein targets (Table S7). Samples were analyzed using LC-MS/MS after being spiked with 50 fmol/uL PRTC standards (88320, Pierce Retention Time Calibration Mixture, Thermo Fisher, MA). A nanoflow ultra high performance liquid chromatograph and nanoelectrospray orbitrap mass spectrometer (RSLCnano and Q Exactive plus, Thermo) were used for LC-MS/MS. The sample was loaded onto a pre-column (C18 PepMap100, 2 cm length×100 μm ID packed with C18 reversed-phase resin, 5 μm particle size, 100 Å pore size) and washed for 8 minutes with aqueous 2% acetonitrile and 0.1% formic acid. Trapped peptides were eluted onto the analytical column, (C18 PepMap100, 25 cm length×75 μm ID, 2 μm particle size, 100 Å pore size, Thermo). A 120-minute gradient was programmed as: 95% solvent A (aqueous 2% acetonitrile+0.1% formic acid) for 8 minutes, solvent B (aqueous 90% acetonitrile+0.1% formic acid) from 5% to 38.5% in 90 minutes, then solvent B from 50% to 90% B in 7 minutes and held at 90% for 5 minutes, followed by solvent B from 90% to 5% in 1 minute and re-equilibration for 10 minutes using a flow rate of 300 nl/min. Spray voltage was 1900 V. Capillary temperature was 275° C. S lens RF level was set at 40. Top 16 tandem mass spectra were collected in a data-dependent manner. The resolution for MS and MS/MS were set at 70,000 and 17,500 respectively. Dynamic exclusion was 15 seconds for previously sampled peaks.

### Data Processing

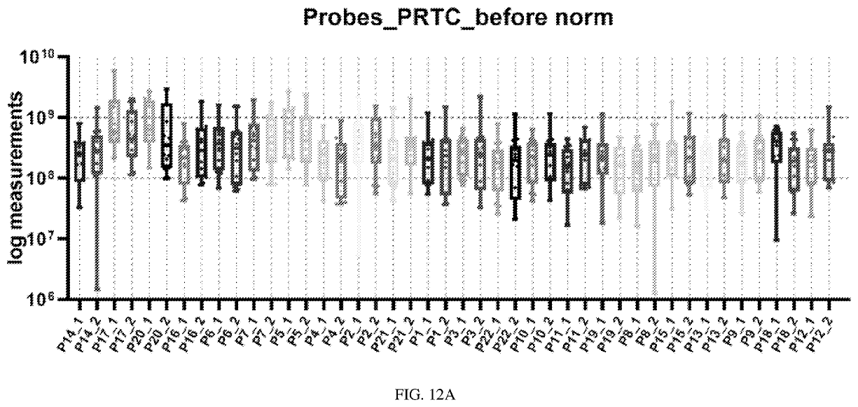

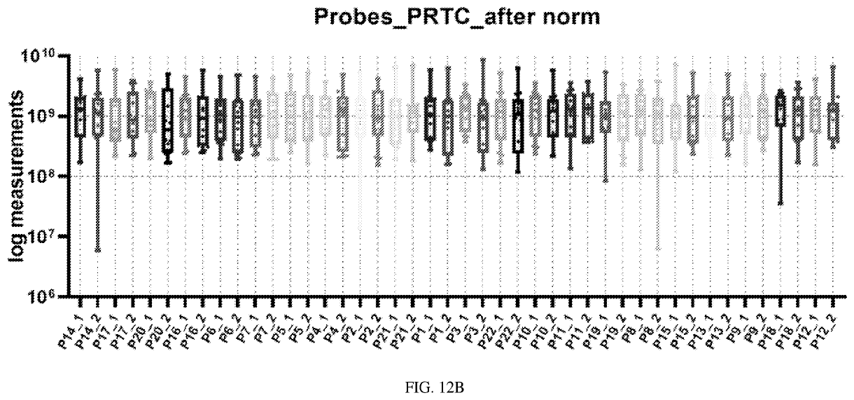

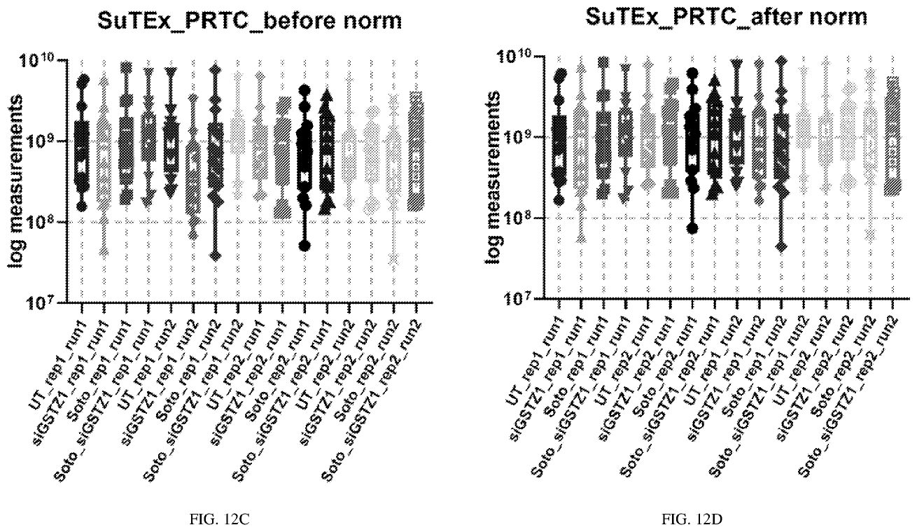

MS/MS data were collected and searched with MaxQuant⁵⁸ against human entries in the UniProt human database (2021). Trypsin/P was set as the digestion enzyme, and carbamidomethyl (C) was selected as a fixed modification. A maximum of 2 missed cleavages was allowed. Label-free quantification (LFQ) was enabled with LFQ min ratio count set to 1. Precursor and fragment ion tolerance were set to 20 ppm and 0.05 Da, respectively. Protein FDR was set as 0.01. Re-quantify and match between runs (2 min) were allowed for peptide identification. Q-value<0.05, reverse counterparts, and contaminants were filtered before further data analysis. Signal intensities of PRTC peptides were extracted using Skyline⁵⁹. The precursor ion signal of each PRTC peptide was summed to generate a value for total PRTC signal in each MS run (FIGS. 12A-12D). Protein intensity signals were normalized to total PRTC signals and then subjected to log 2 transformation. Enriched proteins were selected by comparing the control probe 22 group. Two-tailed Welch's t-test was used to compare two probe groups. Probe-selective proteins were selected based on cross-comparisons of a given probe with the other probes. Enrichment score was calculated based on the difference between the average of a given protein signal intensity for one probe and the average of that signal intensity across all other probes plus one standard deviation (SD). Enrichment score>2, and log 2 ratio>2.32 over the control probe were applied as cutoffs for identifying high-confidence targets.

### Protein Tyrosine Reactivity Profiling

H1792 cells were transfected with siRNA and after 72 h treated with DMSO and sotorasib for another 24 h. Cell lysates were prepared and diluted to 2 mg/mL in PBS. Samples were treated with HHS-482 (a SuTEx probe) at 25 μM for 1 h at room temperature. Labeled proteomes were subjected to CuAAC conjugation with Biotin-Azide conjugate (200 μM) by mixing with TCEP (1 mM), TBTA (100 μM), and CuSO₄ (1 mM). After 1 h incubation, resulting mixtures were subjected to MeOH: CHCl₃ extraction as described above. Protein discs were reconstituted with urea buffer supplemented with 10% SDS. Samples were processed with NeutrAvidin beads, on-bead digestion with trypsin, and ZIPTIP purification as described above. Samples were dried in vacuum and prepared for LC-MS/MS analysis. Proteins signals were standardized by PRTC peptide signals and log 2 transformed. siGSTZ1 or/and sotorasib treatment groups were compared to the DMSO-treated group, and enriched proteins were selected by applying the following cutoffs: log 2 ratio≤−0.585 or ≥0.585 and one-sample t test p value<0.05.

### Lung Cancer Patient Survival Analysis

The Kaplan Meier plotter was used to assess the correlation of GSTZ1 gene expression with LUAD and LUSQ patient survival. mRNA Chip seq data was selected. Plotting parameters were defined as: Affy ID 209531_at; patient survival month threshold was 120 months; Auto selected best cutoff and censored at threshold were checked. Other settings remain as default values. For LUAD patient survival analysis, all datasets were included, and the analysis was run on 719 patients; for LUSQ patient survival analysis, GSE4573 dataset was selected, and the analysis was run on 130 patients.

### Molecular Modeling

We performed molecular docking studies to reveal the predicted binding interactions of fragment 17 with GSTZ1. The co-crystal structure of GSTZ1 and glutathione (PDB ID: 1FW1) enabled the docking studies³². The protein was prepared using the Protein Preparation Wizard implemented in the Maestro 11.1 (Schrödinger Release 2021-1) interface. Water and metal molecules were removed. From the refined structure, receptor grids were generated using default values and it was observed that the docked model of glutathione agreed with the reported crystal structures coordinate (thus, validating our model). The probe 17 and the BioCore of probe 17 were then docked in the created grid using Glide^{31,60} in standard precision (SP) mode and without any constraints.

### GSTZ1 Enzymatic Activity Assay

The GSTZ1 activity assay with its substrate 4-Chloro-7-nitrobenzofurazan (CNBF) was read at 420 nm, which indicates the absorbance of CNBF-GSH adducts as previously reported⁶¹. For experiments without UV irradiation, the reaction was started by adding DMSO or probe 17, 10 μg of recombinant GSTZ1 proteins, 0.2 mM of CNBF, and 2 mM of GSH at room temperature in a final volume of 100 μl HEPES buffer at pH 5.5. For UV irradiation experiments, GSTZ1 protein was incubated with DMSO or probe 17 for 10 minutes under UV light. Each reaction was performed as at least two biological replicates. Spectrophotometry measurements were recorded using an M5 Spectramax plate reader (Molecular devices). Data was processed and analyzed using Excel and GraphPad Prism 9.0. Optical density (OD) readouts of GSTZ1-catalyzed reactions were corrected by subtracting measurements of non-enzymatic reactions. Relative GSTZ1 activity was calculated by normalizing corrected OD readouts of GSTZ1-catalyzed reactions with probe 17 to that of GSTZ1-catalyzed reactions without probe 17. For assays with UV irradiation, recombinant GSTZ1 was produced in-house. To this end, 6His-GFP tag full length human GSTZ1 (Uniprot ID: 043708) with TEV protease cleavage site in pET28-a-(+) vector (GeneScript) was transformed into BL21 DE3 cells (Cat #EC0114, ThermoFisher). Cells were grown in LB Broth, Miller media (Cat.BP1426-2, Fisher Bioreagents) until OD of 0.5-0.8 at 37° C., then induced with 0.1 M IPTG, and harvested after overnight growth at 18° C. by centrifuge at 6000 g at 4° C. The cell pellet was lysed in buffer A (50 mM HEPES pH 8.0, 300 mM NaCl, 20 mM Imidazole, 0.5 mM TCEP) with 0.01% Triton X-100. Cells were homogenized at 1100 psi three times by homogenizer (APV-2000 Invensys), centrifuged at 17000 g for 45 minutes at 4° C., then 6His-GFP tag GSZ1 was purified from supernatant first by manual packed nickel affinity column (Column resin, Ni-NTA superflow 30410, Qiagen) using gradient elution with buffer B (50 mM HEPES pH 8.0, 300 mM NaCl, 500 mM Imidazole, 0.5 mM TCEP). After TEV protease cleavage overnight at 4° C., untagged GSTZ1 was purified by a second nickel affinity column. The purity of GSTZ1 was further improved by size exclusion chromatography using Superdex 75 60/26 column (Cat. 28-9893-34, GE Life Sciences) in buffer C (50 mM HEPES pH 7.5, 50 mM NaCl, 2 mM DTT). The final protein at >99% purity was concentrated to 10 mg/mL, flash frozen with liquid nitrogen, and stored at −80° C. until further use.

### GO and Pathway Analysis

GO analysis was performed using The Database for Annotation, Visualization and Integrated Discovery (DAVID). All significantly changed genes were used as the input for the pathway enrichment in Metascape⁶². Reactome gene and hallmark gene set analysis were enabled with p value cutoff being set as 0.05. Other parameters remain as default values.

### Statistical Analysis

Unless otherwise indicated, data were obtained from at least three independent experiments and statistical analyses were performed using GraphPad Prism 9.0. Error bars represent±standard deviation (SD). The differences between sets of data were compared using unpaired Student's t-test or one-way ANOVA with significant level p=0.05. Asterisks were displayed as *P<0.05, **P<0.01, ***P<0.001.

### Chemical Synthesis

All reagents were purchased from commercial suppliers and were used without further purification. The reactions were either monitored by silica gel chromatography or analytical LC-MS. Thin layer chromatography was performed on Kieselgel 60 F₂₅₄ glass plates pre-coated with a 0.25 mm thickness of silica gel and the TLC plates were visualized with UV light and/or by staining with ninhydrin or potassium permanganate solutions. Normal phase column chromatography was performed on a Biotage Selekt automated flash system. The compounds were loaded onto pre-filled cartridges filled with KP-Sil 50 μm irregular silica. Some of the final products were isolated by reverse phase HPLC using Waters HPLC system with UV detector, with Atlantis T3 OBD Prep Column, 19 mm ID×150 mm length, 5 μm particle size, and 100 Å pore size. Compounds were eluted using a gradient elution of 90/10 to 0/100 A/B over 20 min at a flow rate of 20.0 mL/min, where solvents A and B were water (+0.1% trifluoroacetic acid) and acetonitrile, respectively.

The structures of all compounds were verified via 1H NMR, and LC-MS, and the purities of the isolated products were determined using an LC-MS instrument (Agilent 1290 Infinity series LC with single quadrupole MSD system, AP-ESI Ion Source) equipped with Kinetex® LC 2.1 mm×50 mm column packed with 1.7 μm C₁₈ particles with 100 Å pore size, Ea (Phenomenex) column. Elution was performed using the following conditions: 2% (v/v) acetonitrile (+0.1% FA) in 98% (v/v) H₂O (+0.1% FA), ramped to 98% acetonitrile over 4.0 min, and holding at 98% acetonitrile for 0.5 min with a flow rate of 0.6 mL/min; UV absorption was detected from 200 to 950 nm using a diode array detector. The purity of each compound was ≥95% based on this analysis.

NMR spectra were recorded at ambient temperature on a 500 MHz Bruker NMR spectrometer in DMSO-d6/CDCl₃. All 1H NMR data are reported in parts per million (ppm) downfield of TMS and were measured relative to the signals for dimethyl sulfoxide (2.50 ppm) and deuterated chloroform (7.22 ppm). Data for 1H NMR are reported as follows: chemical shift (8, ppm), multiplicity (s=singlet, d=doublet, t=triplet, q=quartet, m=multiplet), integration, and coupling constant (Hz). NMR data was analyzed and processed by using MestReNova software.

### Compound Synthesis and Characterization

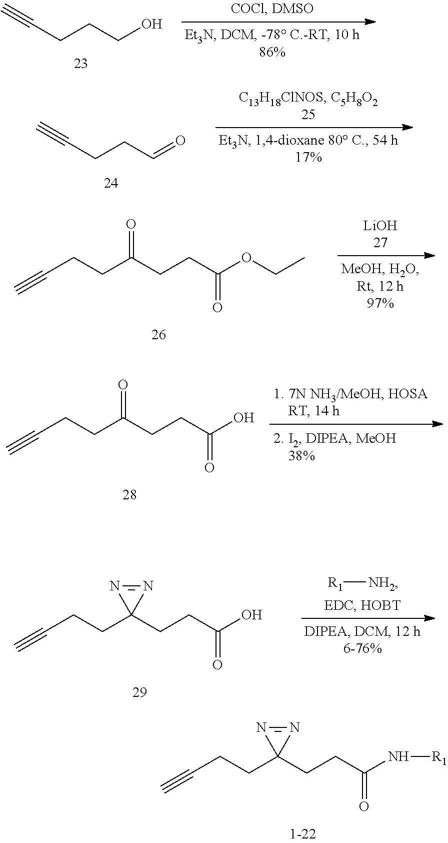

Pent-4-ynal 24 was generated by Swern oxidation of the commercially available 23 in 86% yield. The ethyl 4-oxooct-7-ynoate 26 was accessed through the Stetter reaction of ethyl acrylate 25 and pent-4-ynal 24, which facilitated the hydrolysis of 26 to provide 28 in 97% yield. Subsequently, the diazirine was introduced at the ketone of 28 in a two-step procedure to first generate the diaziridine, which was oxidized to afford the diazirine 29 in 38% yield. The probes were accessed via the amide coupling of 29 with the commercially available amines displayed in FIG. 1.
Pent-4-ynal (24)

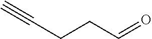

To a stirred solution of DCM (90 mL) and oxalyl chloride (4.53 g, 35.66 mmol, 1.50 equiv.) was added DMSO (5.58 g, 71.42 mmol, 3.00 equiv.) at −78° C. After 15 min of stirring, 4-pentynol (2.00 g, 23.78 mmol, 1.00 equiv.) in DCM (30 mL) was added dropwise to the reaction mixture and stirred for 15 min. Et₃N (10.83 g, 106.99 mmol, 4.50 equiv.) was slowly added to the reaction mixture and left to stir for an additional 15 min, then the reaction was warmed to RT overnight and quenched with H₂O. The aqueous layer was extracted with DCM, and the combined organics was washed with H₂O, brine and dried over Na₂SO₄, and concentrated under reduced pressure to give crude 24 as a dark brown liquid (1.68 g, 86%) that was used without further purification.

¹H NMR (500 MHZ, CDCl₃): δ 9.80 (t, J=1.1 Hz, 1H), 2.73-2.68 (m, 2H), 2.54-2.48 (m, 2H), 1.99 (t, J=2.7 Hz, 1H).

### Ethyl 4-oxooct-7-ynoate (26)

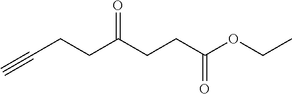

A solution of freshly synthesized 24 (1.50 g, 18.27 mmol, 1.00 equiv.) and ethyl acrylate (3.66 g, 36.54 mmol, 2.00 equiv.) in 1,4-dioxane (21.90 mL) was added dropwise over a period of 2 h to a suspension of 3-benzyl-5-(2-hydroxyethyl)-4-methylthiazol-3-ium chloride (0.739 g, 2.74 mmol, 0.15 equiv.), Et₃N (1.29 g, 12.79 mmol, 0.70 equiv.) and ethyl acrylate (3.66 g, 36.54 mmol, 2.00 equiv.) in 1,4-dioxane (26.26 mL) at 80° C. under an atmosphere of argon. The mixture was then stirred for 54 h at 80° C. when TLC indicated complete conversion. The volatiles were removed under reduced pressure, resuspended in DCM, washed with 10% H₂SO₄, sat.aq. NaHCO₃ and brine and dried with Na₂SO₄. The volatiles were removed under reduced pressure to give crude 26. The residue was purified by flash column chromatography (silica gel, 0% to 30% EtOAc: hexanes) to give 26 as a light brown oil (0.550 g, 17%).

¹H NMR (500 MHZ, CDCl₃): δ 4.13 (q, J=7.1 Hz, 2H), 2.76-2.70 (m, 4H), 2.62-2.58 (m, 2H), 2.47 (d, J=2.7 Hz, 2H), 1.95 (t, J=2.7 Hz, 1H), 1.25 (t, J=7.1 Hz, 3H).

### 4-oxooct-7-ynoic acid (28)

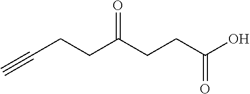

To a stirred solution of 26 (0.426 g, 2.34 mmol, 1.00 equiv.) in MeOH (18.04 mL) and H₂O (0.215 mL) was added LiOH (0.280 g, 11.69 mmol, 5.00 equiv.) and stirred overnight at RT. After completion, which was monitored by TLC (KMNO₄ stain), the solution was acidified with 1M HCl until a pH of 3 was obtained. The resulting solution was extracted with DCM, and the organics were dried with Na₂SO₄ and concentrated under reduced pressure to give the crude 28 as a brown solid (0.350 g, 97%), which was used without further purification.

¹H NMR (500 MHZ, CDCl₃): δ 2.78-2.69 (m, 4H), 2.67 (ddd, J=6.8, 6.1, 1.2 Hz, 2H), 2.47 (ddd, J=8.3, 6.6, 2.6 Hz, 2H), 1.95 (t, J=2.7 Hz, 1H).

### 3-(3-(but-3-yn-1-yl)-3H-diazirin-3-yl) propanoic acid (29)

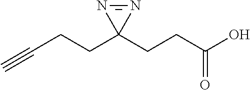

To a pressure flask at −78° C. was added NH₃ (0.602 mL) under an atmosphere of argon. Anhydrous MeOH was added to the vessel to achieve the desired concentration of 7N NH₃ in MeOH. At −78° C., ° a solution of 28 (0.312 mg, 2.02 mmol, 1.00 equiv.) in anhydrous MeOH (2.54 mL) was added to the solution of 7N NH₃ in MeOH. The resulting solution was transferred to an ice bath and stirred under an atmosphere of argon for 3 h at 0° C. when hydroxyl-amine-O-sulfonic acid (0.320 g, 2.83 mmol, 1.40 equiv.) in MeOH (2.54 mL) was added dropwise to the reaction mixture at 0° C. The reaction mixture was stirred at the same temperature for an additional 1 h and allowed to warm to RT and stirred for 14 h or until TLC indicated completion of the reaction. The resulting suspension was evaporated under reduced pressure, resuspended in MeOH and the solid was filtered and washed several times with MeOH. The combined filtrate was evaporated, resuspended in anhydrous MeOH (18.10 mL), then cooled to 0° C. under an atmosphere of argon. Anhydrous DIPEA (0.583 g, 4.51 mmol, 2.23 equiv.) was added and stirred at 0° C. for 5 min (at this point the flask was protected from light), followed by the portion wise addition of iodine (0.514 g, 2.02 mmol, 1.00 equiv.) until a dark brown color change persisted for more than 30 min, indicating complete oxidation of diaziridine. The solution was then diluted with EtOAc, wash with IN HCl (until a pH of 3 was attained), sat.aq. Na₂S₂O₃ (×3 or until organic phase is clarified) and brine. The combined organics were dried over Na₂SO₄, and the volatiles were removed under reduced pressure to give crude 29. The residue was purified by flash column chromatography (silica gel, 0% to 35% EtOAc: hexanes) to give 29 as a colorless oil (0.129 g, 38%).

¹H NMR (500 MHZ, CDCl₃): δ 2.18 (dd, J=8.1, 7.2 Hz, 2H), 2.05-1.98 (m, 3H), 1.82 (dd, J=8.1, 7.2 Hz, 2H), 1.66 (t, J=7.3 Hz, 2H).

### General Procedure A

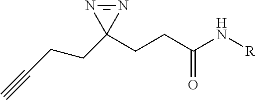

To a flame dried microwave vial containing 29 (1.00 equiv., 0.048-0.100 mmol) in DCM (0.25 molar), was added DIPEA (3.00 equiv., 0.180-0.397 mmol) EDC (1.50 equiv., 0.072-0.090 mmol) and HOBT (1.50 equiv., 0.072-0.090 mmol). The reaction mixture was stirred at RT for 15 min when commercially available amine (1.10 equiv., 0.059-0.066 mmol) was added and stirred overnight (or when TLC indicated completion of the reaction) at RT and under an atmosphere of argon. The reaction mixture was diluted with DCM and washed first with sat.aq. NH₄Cl, followed by sat.aq. NaHCO₃ and dried over Na₂SO₄. The volatiles were removed under reduced pressure to give the crude products that were purified by flash column chromatography (silica gel) or preparative HPLC.

### 1-(4-(1H-benzo[d]imidazol-2-yl)piperidin-1-yl)-3-(3-(but-3-yn-1-yl)-3H-diazirin-3-yl)propan-1-one (1)

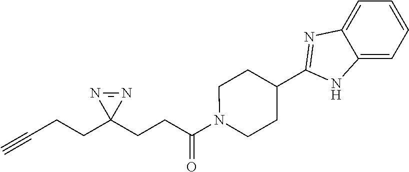 [SMILES: C#CCCC1(CCC(=O)N2CCC(c3nc4ccccc4[nH]3)CC2)N=N1]

Probe 1 was prepared as described in General Procedure A: purified by prep HPLC to afford 1 as a transparent oil (0.007 g, 37%).

¹H NMR (500 MHZ, CDCl₃): δ 7.59-7.55 (m, 2H), 7.24 (dd, J=6.0, 3.1 Hz, 2H), 4.68-4.61 (m, 1H), 3.93-3.86 (m, 1H), 3.19 (tdd, J=14.6, 8.2, 3.3 Hz, 2H), 2.78 (td, J=13.2, 12.8, 2.9 Hz, 1H), 2.10 (dt, J=11.4, 3.7 Hz, 4H), 2.02 (td, J=7.4, 2.7 Hz, 2H), 1.97 (t, J=2.6 Hz, 1H), 1.95-1.73 (m, 4H), 1.66 (t, J=7.6 Hz, 2H). LCMS (m/z): 350 (M+1). Rₜ: 5.732 min.

### 3-(3-(but-3-yn-1-yl)-3H-diazirin-3-yl)-N-(3,3-dimethyl-5-((2-methylpyrazolo[1,5-a]pyrazin-4-yl)oxy)cyclohexyl)propanamide (2)

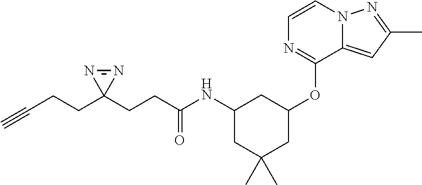 [SMILES: C#CCCC1(CCC(=O)NC2CC(Oc3nccn4nc(C)cc34)CC(C)(C)C2)N=N1]

Probe 2 was prepared as described in General Procedure A: Purified by flash chromatography (silica gel, 0% to 50% EtOAc/hexanes) to afford 2 as a pale-yellow oil (0.018 g, 71%).

¹H NMR (500 MHZ, CDCl₃): δ 7.87 (dd, J=4.8, 0.9 Hz, 1H), 7.23 (d, J=4.8 Hz, 1H), 6.45 (s, 1H), 5.43 (u, J=11.4, 4.4 Hz, 1H), 5.32 (d, J=8.2 Hz, 1H), 4.22-4.07 (m, 1H), 2.56 (ddq, J=10.4, 3.8, 2.0 Hz, 1H), 2.46 (s, 3H), 2.04-1.98 (m, 2H), 1.96 (t, J=2.6 Hz, 1H), 1.93 (dt, J=4.0, 1.9 Hz, 1H), 1.90-1.81 (m, 4H), 1.73 (ddt, J=12.5, 3.9, 1.9 Hz, 1H), 1.63 (t, J=7.4 Hz, 2H), 1.33 (t, J=12.0 Hz, 1H), 1.27-1.16 (m, 2H), 1.08 (s, 3H), 1.02 (s, 3H). LCMS (m/z): 323 (M+1), Rₜ: 9.234 min.

### 3-(3-(but-3-yn-1-yl)-3H-diazirin-3-yl)-N-(4-((4-methylpiperazin-1-yl)methyl)phenyl)propenamide (3)

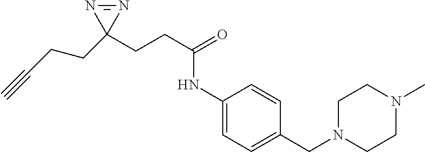 [SMILES: C#CCCC1(CCC(=O)Nc2ccc(CN3CCN(C)CC3)cc2)N=N1]

Probe 3 was prepared as described in General Procedure A: purified by prep HPLC to afford 3 as an oily acetate salt (0.008 g, 38%).

¹H NMR (500 MHZ, CDCl₃): δ 7.45 (d, J=8.2 Hz, 2H), 7.42 (s, 1H), 7.24 (d, J=8.2 Hz, 2H), 3.50 (s, 2H), 2.64 (m, J=57.2 Hz, 7H), 2.41 (s, 3H), 2.12 (dd, J=8.3, 6.7 Hz, 2H), 2.04 (dd, J=7.5, 2.6 Hz, 2H), 1.98 (t, J=2.6 Hz, 1H), 1.93 (dd, J=8.3, 6.7 Hz, 2H), 1.68 (t, J=7.4 Hz, 2H). LCMS (m/z): 354 (M+1). Rₜ: 4.773 min.

### 3-(3-(but-3-yn-1-yl)-3H-diazirin-3-yl)-1-(4-((4-(2-methoxyphenyl)-4H-1,2,4-triazol-3-yl)methyl)piperazin-1-yl)propan-1-one (4)

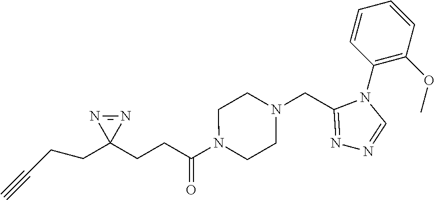 [SMILES: C#CCCC1(CCC(=O)N2CCN(Cc3nncn3-c3ccccc3OC)CC2)N=N1]

Probe 4 was prepared as described in General Procedure A: Purified by prep HPLC to afford 4 as a yellow oil (0.012 g, 47%).

¹H NMR (500 MHZ, CDCl₃): δ 6.53 (ddd, J=8.4, 7.5, 1.7 Hz, 1H), 6.38 (dd, J=8.0, 1.7 Hz, 1H), 6.33 (s, 1H), 6.16-6.09 (m, 2H), 2.86 (s, 3H), 2.69 (s, 2H), 2.45 (t, J=5.1 Hz, 2H), 2.30-2.23 (m, 2H), 1.47 (t, J=5.0 Hz, 2H), 1.37 (t, J=5.1 Hz, 2H), 1.11-1.00 (m, 5H), 0.90-0.84 (m, 2H), 0.70 (t, J=7.4 Hz, 2H). LCMS (m/z): 422 (M+1). Rₜ: 6.978 min.

### 3-(3-(but-3-yn-1-yl)-3/-diazirin-3-yl)-1-(3-((3-chloro-5-(trifluoromethyl)pyridin-2-yl)oxy)pyrrolidin-1-yl)propan-1-one (5)

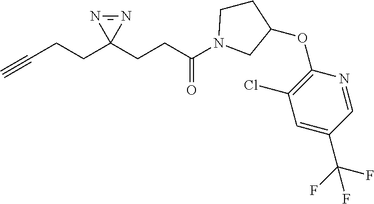 [SMILES: C#CCCC1(CCC(=O)N2CCC(Oc3ncc(C(F)(F)F)cc3Cl)C2)N=N1]

Probe 5 was prepared as described in General Procedure A: Purified by flash chromatography (silica gel, 0% to 50% EtOAc/hexanes) to afford 5 as a transparent oil (0.014 g, 57%).

¹H NMR (500 MHZ, CDCl₃): δ 8.32 (dd, J=10.4, 1.2 Hz, 1H), 7.86 (dd, J=12.2, 2.2 Hz, 1H), 5.68 (dtt, J=17.4, 4.4, 2.0 Hz, 1H), 3.86-3.70 (m, 2H), 3.70-3.58 (m, 2H), 2.41-2.16 (m, 2H), 2.12-1.95 (m, 5H), 1.91-1.85 (m, 2H), 1.67 (td, J=7.4, 5.7 Hz, 2H). ¹⁹F {1H} NMR (471 MHZ, CDCl₃) δ−61.53 (s, 3F). LCMS (m/z): 415 (M+1). Rₜ: 9.300 min.

### 3-(3-(but-3-yn-1-yl)-3H-diazirin-3-yl)-1-(4-((4-(thiophen-2-yl)thiazol-2-yl)methyl)piperazin-1-yl)propan-1-one (6)

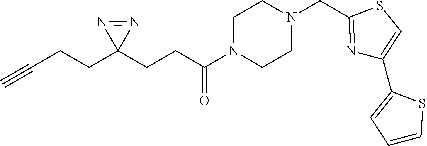 [SMILES: C#CCCC1(CCC(=O)N2CCN(Cc3nc(-c4cccs4)cs3)CC2)N=N1]

Probe 6 was prepared as described in General Procedure A: Purified by flash chromatography (silica gel, 0% to 50% EtOAc/hexane) to afford 6 as a yellow solid (0.013 g, 39%).

¹H NMR (500 MHZ, CDCl₃): δ 7.42 (d, J=2.4 Hz, 1H), 7.37 (s, 1H), 7.29 (d, J=5.0 Hz, 1H), 7.06 (dd, J=5.1, 3.6 Hz, 1H), 3.88-3.46 (m, 4H), 2.92-2.51 (m, 4H), 2.02 (ddd, J=10.0, 6.4, 2.6 Hz, 4H), 1.98 (t, J=2.6 Hz, 1H), 1.86 (dd, J=8.7, 6.4 Hz, 2H), 1.65 (t, J=7.4 Hz, 2H), 1.54 (t, J=7.2 Hz, 1H), 1.45-1.41 (m, 1H). LCMS (m/z): 414 (M+1). Rt: 8.708 min.

### 3-(3-(but-3-yn-1-yl)-3H-diazirin-3-yl)-N-(5-isobutyl-3,3-dimethyl-4-oxo-2,3,4,5-tetrahydrobenzo[b][1,4]oxazepin-7-yl)propanamide (7)

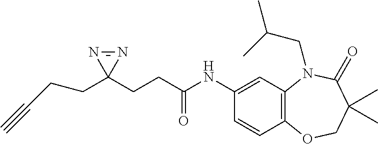 [SMILES: C#CCCC1(CCC(=O)Nc2ccc3c(c2)N(CC(C)C)C(=O)C(C)(C)CO3)N=N1]

Probe 7 was prepared as described in General Procedure A: Purified by flash chromatography (silica gel, 0% to 40% EtOAc/hexanes) to afford 7 as a white solid (0.015 g, 61%).

¹H NMR (500 MHZ, CDCl₃): δ 7.76 (d, J=2.6 Hz, 1H), 7.58 (dd, J=8.9, 2.7 Hz, 1H), 6.80 (d, J=9.0 Hz, 1H), 3.77 (s, 2H), 3.70 (d, J=6.3 Hz, 2H), 2.13-2.06 (m, 3H), 2.06-1.99 (m, 3H), 1.90 (dd, J=8.5, 6.7 Hz, 2H), 1.67 (t, J=7.4 Hz, 2H), 1.38 (s, 6H), 1.04 (d, J=6.7 Hz, 6H). LCMS (m/z): 411 (M+1). Rₜ: 9.335 min.

### 3-(3-(but-3-yn-1-yl)-3H-diazirin-3-yl)-N-(4-(3-cyclopropyl-6-oxopyridazin-1(6H)-yl)cyclohexyl)propenamide (8)

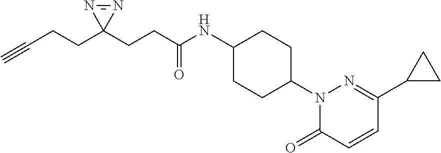 [SMILES: C#CCCC1(CCC(=O)NC2CCC(n3nc(C4CC4)ccc3=O)CC2)N=N1]

Probe 8 was prepared as described in General Procedure A: Purified by flash chromatography (silica gel, 0% to 80% EtOAc/hexanes) to afford 8 as a transparent oil (0.022 g, 95%).

¹H NMR (500 MHZ, CDCl₃): δ 6.96 (d, J=9.5 Hz, 1H), 6.84 (d, J=9.5 Hz, 1H), 5.68 (d, J=7.6 Hz, 1H), 4.88 (tt, J=10.8, 3.9 Hz, 1H), 4.15 (dt, J=7.4, 3.5 Hz, 1H), 2.08-1.81 (m, 12H), 1.76 (dt, J=14.4, 3.8 Hz, 4H), 1.70-1.60 (m, 6H), 1.05-0.95 (m, 2H), 0.92-0.79 (m, 2H). LCMS (m/z): 382 (M+1). Rₜ: 8.756 min.

### 4-(4-(3-(3-(but-3-yn-1-yl)-3H-diazirin-3-yl) propanoyl)piperazin-1-yl)-N-cyclohexylbenzamide (9)

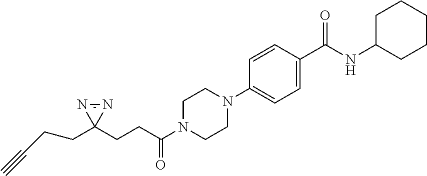 [SMILES: C#CCCC1(CCC(=O)N2CCN(c3ccc(C(=O)NC4CCCCC4)cc3)CC2)N=N1]

Probe 9 was prepared as described in General Procedure A: Purified by flash chromatography (silica gel, 25% to 100% EtOAc/hexane) to afford 9 as a white solid (0.016 g, 61%). LCMS (m/z): 436 (M+1). R: 9.080 min.

### 3-(3-(but-3-yn-1-yl)-3H-diazirin-3-yl)-N-((4-(3-fluorophenyl)tetrahydro-2H-pyran-4-yl)methyl)propenamide (10)

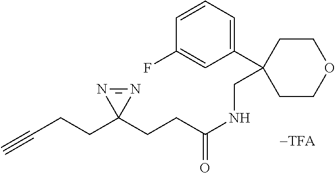 [SMILES: C#CCCC1(CCC(=O)NCC2(c3cccc(F)c3)CCOCC2)N=N1]

Probe 10 was prepared as described in General Procedure A: purified by prep HPLC to afford the trifluoroacetate salt of 10 as a pale-yellow solid (0.007 g, 25%). 1H NMR (500 MHZ, CDCl₃): δ 7.42-7.34 (m, 1H), 7.09 (ddd, J=7.9, 1.8, 1.0 Hz, 1H), 7.04-6.96 (m, 2H), 4.93 (d, J=5.6 Hz, 1H), 3.84 (ddd, J=11.8, 6.9, 3.4 Hz, 2H), 3.60 (ddd, J=11.4, 7.7, 3.3 Hz, 2H), 3.50 (d, J=6.4 Hz, 2H), 2.07-1.97 (m, 4H), 1.95 (t, J=2.6 Hz, 1H), 1.91-1.85 (m, 2H), 1.82-1.79 (m, 4H), 1.61 (t, J=7.4 Hz, 2H). 19F NMR (471 MHz, CDCl₃) δ−111.61 (s, 1F). LCMS (m/z): 358 (M+1). Rₜ: 8.903 min.

### N-(1-(3-(3-(but-3-yn-1-yl)-3H-diazirin-3-yl)propanoyl)piperidin-4-yl)-4-fluorobenzamide (11)

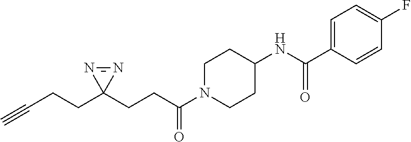 [SMILES: C#CCCC1(CCC(=O)N2CCC(NC(=O)c3ccc(F)cc3)CC2)N=N1]

Probe 11 was prepared as described in General Procedure A: Purified by flash chromatography (silica gel, 0% to 50% EtOAc/hexanes) to afford 11 as a white solid (0.007 g, 31%).

¹H NMR (500 MHZ, CDCl₃): δ 7.79-7.74 (m, 2H), 7.14-7.09 (m, 2H), 5.98 (d, J=7.8 Hz, 1H), 4.68-4.54 (m, 1H), 3.78 (dd, J=11.7, 7.0 Hz, 1H), 3.20-3.09 (m, 1H), 2.80-2.70 (m, 1H), 2.10-2.00 (m, 5H), 1.98 (t, J=2.6 Hz, 1H), 1.87 (td, J=7.6, 4.1 Hz, 2H), 1.70-1.61 (m, 4H), 1.45-1.32 (m, 2H). 19F NMR (471 MHz, CDCl₃) δ−107.81 (s, 1F). LCMS (m/z): 371 (M+1). Rₜ: 8.718 min.

### 3-(3-(but-3-yn-1-yl)-3H-diazirin-3-yl)-N-(4-((5-fluoropyridin-2-yl)oxy)tetrahydrofuran-3-yl)propenamide (12)

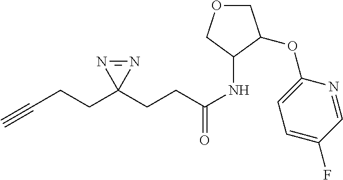 [SMILES: C#CCCC1(CCC(=O)NC2COCC2Oc2ccc(F)cn2)N=N1]

Probe 12 was prepared as described in General Procedure A: Purified by flash chromatography (silica gel, 25% to 100% EtOAc/hexane) to afford 12 as an off-white solid (0.013 g, 39%).

¹H NMR (500 MHZ, CDCl₃): δ 7.93 (d, J=3.1 Hz, 1H), 7.35 (ddd, J=9.0, 7.5, 3.1 Hz, 1H), 6.76 (dd, J=9.0, 3.5 Hz, 1H), 6.08 (d, J=6.7 Hz, 1H), 5.26 (dt, J=5.2, 2.6 Hz, 1H), 4.25 (dd, J=10.6, 5.4 Hz, 1H), 4.17 (dd, J=9.6, 5.2 Hz, 1H), 3.82 (dd, J=10.6, 3.0 Hz, 1H), 3.75 (dd, J=9.6, 3.1 Hz, 1H), 2.06-1.93 (m, 5H), 1.88-1.83 (m, 2H), 1.64 (t, J=7.3 Hz, 2H). 19F NMR (471 MHZ, CDCl₃) δ−138.22. LCMS (m/z): 347 (M+1). Rₜ: 2.534 min.

### 3-(3-(but-3-yn-1-yl)-3H-diazirin-3-yl)-N-(1-(3-(tert-butyl)-[1,2,4]triazolo[4,3-b]pyridazin-6-yl)azetidin-3-yl)-N-methylpropanamide (13)

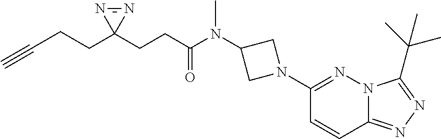 [SMILES: C#CCCC1(CCC(=O)N(C)C2CN(c3ccc4nnc(C(C)(C)C)n4n3)C2)N=N1]

Probe 13 was prepared as described in General Procedure A: Purified by flash chromatography (silica gel, 0% to 10% MeOH/DCM) to afford 13 as a light brown sticky solid (0.024 g, 98%).

¹H NMR (500 MHZ, CDCl₃): δ 8.07 (d, J=9.9 Hz, 1H), 6.64 (d, J=9.8 Hz, 1H), 5.36 (tt, J=8.0, 5.7 Hz, 1H), 4.40 (t, J=8.7 Hz, 2H), 4.18-4.11 (m, 2H), 3.09 (s, 3H), 2.11-2.02 (m, 4H), 1.99 (t, J=2.7 Hz, 1H), 1.93-1.88 (m, 2H), 1.68 (t, J=7.4 Hz, 2H), 1.58 (s, 9H). LCMS (m/z): 409 (M+1). Rₜ: 8.769 min.

### 3-(3-(but-3-yn-1-yl)-3H-diazirin-3-yl)-1-(4-(((2,5-dimethylpyrazolo[1,5-a]pyrimidin-7-yl)oxy)methyl)piperidin-1-yl)propan-1-one (14)

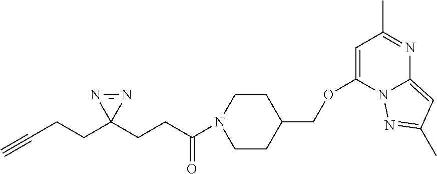 [SMILES: C#CCCC1(CCC(=O)N2CCC(COc3cc(C)nc4cc(C)nn34)CC2)N=N1]

Probe 14 was prepared as described in General Procedure A: Purified by flash Prep HPLC to afford 14 as a pale yellow oil (0.011 g, 45%).

¹H NMR (500 MHZ, CDCl₃): δ 6.29 (s, 1H), 5.95 (s, 1H), 4.69 (ddt, J=13.4, 4.6, 2.3 Hz, 1H), 4.20-4.07 (m, 2H), 3.84 (ddt, J=13.7, 4.6, 2.4 Hz, 1H), 3.09-3.01 (m, 1H), 2.53 (s, 3H), 2.49 (s, 3H), 2.10-2.01 (m, 6H), 1.98 (t, J=2.6 Hz, 1H), 1.86 (ddd, J=9.2, 6.4, 1.8 Hz, 2H), 1.67 (t, J=7.4 Hz, 2H), 1.34-1.21 (m, 4H). LCMS (m/z): 409 (M+1). R: 8.071 min.

### 3-(3-(but-3-yn-1-yl)-3H-diazirin-3-yl)-N-(1-(6-(2-chlorophenyl)pyridazin-3-yl)piperidin-4-yl)propenamide (15)

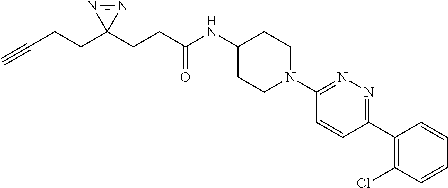 [SMILES: C#CCCC1(CCC(=O)NC2CCN(c3ccc(-c4ccccc4Cl)nn3)CC2)N=N1]

Probe 15 was prepared as described in General Procedure A: Purified by flash chromatography (silica gel, 20% to 100% EtOAc/hexanes) to afford 15 as an off-white solid (0.013 g, 49%).

¹H NMR (500 MHZ, CDCl₃): δ 7.76-7.70 (m, 2H), 7.50-7.46 (m, 1H), 7.41-7.36 (m, 2H), 7.11 (d, J=9.6 Hz, 1H), 4.48 (d, J=13.5 Hz, 2H), 3.28 (m, J=14.0, 11.3, 2.7 Hz, 2H), 2.14-2.05 (m, 3H), 2.02 (td, J=7.5, 2.5 Hz, 2H), 1.99-1.94 (m, 3H), 1.86-1.82 (m, 2H), 1.68-1.57 (m, 4H). LCMS (m/z): 437 (M+1). Rₜ: 8.583 min.

### 3-(3-(but-3-yn-1-yl)-3H-diazirin-3-yl)-1-(4-(6-(4,5-dimethyl-1H-imidazol-1-yl)-2-methylpyrimidin-4-yl)piperazin-1-yl)propan-1-one (16)

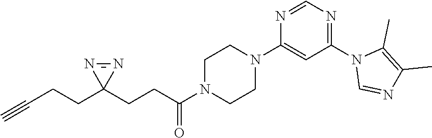 [SMILES: C#CCCC1(CCC(=O)N2CCN(c3cc(-n4cnc(C)c4C)ncn3)CC2)N=N1]

Probe 16 was prepared as described in General Procedure A: Purified by flash chromatography (silica gel, 0% to 10% MeOH/DCM) to afford 16 as a white solid (0.011 g, 44%).

¹H NMR (500 MHz, CDCl₃): δ 7.90 (s, 1H), 6.22 (s, 1H), 3.81-3.71 (m, 4H), 3.65 (dd, J=6.7, 4.0 Hz, 2H), 3.57-3.52 (m, 2H), 2.52 (s, 3H), 2.32 (s, 3H), 2.19 (s, 3H), 2.11-2.03 (m, 4H), 1.98 (t, J=2.6 Hz, 1H), 1.93-1.88 (m, 2H), 1.68 (t, J=7.4 Hz, 2H). LCMS (m/z): 421 (M+1). Rₜ: 6.914 min.

### 3-(3-(but-3-yn-1-yl)-3H-diazirin-3-yl)-1-(4-(((3-methylquinoxalin-2-yl)oxy)methyl)piperidin-1-yl)propan-1-one (17)

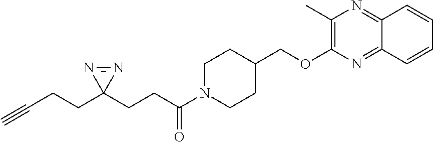 [SMILES: C#CCCC1(CCC(=O)N2CCC(COc3nc4ccccc4nc3C)CC2)N=N1]

Probe 17 was prepared as described in General Procedure A: Purified by flash chromatography (silica gel, 25% to 100% EtOAc/hexanes) to afford 17 as an off white solid (0.017 g, 70%).

¹H NMR (500 MHZ, CDCl₃): δ 7.93 (dd, J=8.2, 1.5 Hz, 1H), 7.78 (dd, J=8.3, 1.5 Hz, 1H), 7.59 (ddd, J=8.3, 6.9, 1.5 Hz, 1H), 7.52 (ddd, J=8.3, 7.0, 1.5 Hz, 1H), 4.68 (ddt, J=13.3, 4.6, 2.3 Hz, 1H), 4.42-4.31 (m, 2H), 3.84 (ddt, J=13.6, 4.6, 2.4 Hz, 1H), 3.05 (td, J=13.1, 12.7, 2.9 Hz, 1H), 2.63 (s, 3H), 2.19-2.10 (m, 1H), 2.10-2.00 (m, 4H), 1.98 (t, J=2.7 Hz, 1H), 1.96-1.78 (m, 4H), 1.68 (t, J=7.3 Hz, 2H), 1.34 (qd, J=12.5, 4.4 Hz, 2H). LCMS (m/z): 406 (M+1). R: 9.292 min.

### 3-(3-(but-3-yn-1-yl)-3H-diazirin-3-yl)-1-(3-(pyrimidin-2-ylamino)azetidin-1-yl)propan-1-one (18)

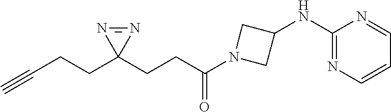 [SMILES: C#CCCC1(CCC(=O)N2CC(Nc3ncccn3)C2)N=N1]

Probe 18 was prepared as described in General Procedure A: Purified by flash chromatography (silica gel, 25% to 100% EtOAc/hexane) to afford 18 as a white solid (0.016 g, 61%).

¹H NMR (500 MHZ, CDCl₃): δ 8.29 (d, J=4.8 Hz, 2H), 6.64 (td, J=4.8, 0.9 Hz, 1H), 6.12 (s, 1H), 4.71 (ddt, J=14.0, 7.4, 5.4 Hz, 1H), 4.45 (ddd, J=8.7, 7.5, 1.1 Hz, 1H), 4.38 (ddd, J=10.5, 7.8, 1.1 Hz, 1H), 3.97 (dd, J=8.9, 5.1 Hz, 1H), 3.89 (dd, J=10.4, 5.4 Hz, 1H), 2.02 (td, J=7.4, 2.6 Hz, 2H), 1.98 (t, J=2.6 Hz, 1H), 1.84 (q, J=2.7, 2.1 Hz, 4H), 1.65 (t, J=7.4 Hz, 2H). LCMS (m/z): 299 (M+1). Rₜ: 2.060 min.

### Methyl-5-(1-(3-(3-(but-3-yn-1-yl)-3H-diazirin-3-yl)propanoyl)piperidin-2-yl)-1,3,4-thiadiazole-2-carboxylate (19)

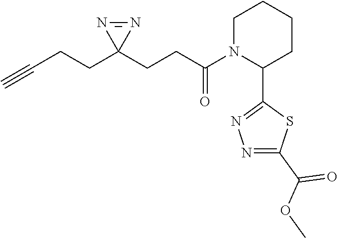 [SMILES: C#CCCC1(CCC(=O)N2CCCCC2c2nnc(C(=O)OC)s2)N=N1]

Probe 19 was prepared as described in General Procedure A: Purified by flash chromatography (silica gel, 0% to 70% EtOAc/hexanes) to afford 19 as a transparent oil (0.007 g, 31%).

¹H NMR (500 MHZ, CDCl₃): δ 6.26-6.19 (m, 1H), 4.03 (s, 3H), 3.71 (dt, J=13.9, 3.2 Hz, 1H), 3.09 (td, J=13.3, 2.8 Hz, 1H), 2.59 (dtd, J=13.9, 5.2, 4.4, 2.6 Hz, 1H), 2.20-2.09 (m, 2H), 2.07-1.97 (m, 3H), 1.93-1.88 (m, 2H), 1.81-1.71 (m, 5H), 1.69 (t, J=7.5 Hz, 2H). LCMS (m/z): 376 (M+1). Rₜ: 2.687 min.

### 2-((1-(3-(3-(but-3-yn-1-yl)-3H-diazirin-3-yl)propanoyl)pyrrolidin-2-yl)methoxy)quinoline-3-carbonitrile (20)

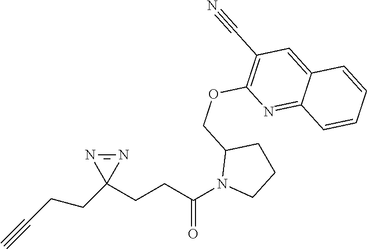 [SMILES: C#CCCC1(CCC(=O)N2CCCC2COc2nc3ccccc3cc2C#N)N=N1]

Probe 20 was prepared as described in General Procedure A: Purified by flash chromatography (silica gel, 0% to 60% EtOAc/hexanes) to afford 20 as a pale yellow solid (0.017 g, 70%).

¹H NMR (500 MHZ, CDCl₃): δ 8.39 (s, 1H), 7.83 (dt, J=8.9, 1.1 Hz, 1H), 7.77-7.72 (m, 2H), 7.46 (ddd, J=8.1, 6.9, 1.2 Hz, 1H), 4.81 (dd, J=10.7, 4.7 Hz, 1H), 4.60-4.52 (m, 2H), 3.63 (ddd, J=9.9, 8.4, 4.1 Hz, 1H), 3.46 (dt, J=9.9, 7.8 Hz, 1H), 2.14-1.91 (m, 9H), 1.89-1.80 (m, 2H), 1.63 (t, J=7.5 Hz, 2H). LCMS (m/z): 402 (M+1). Rₜ: 9.242 min.

### N-(3-(3-(3-(but-3-yn-1-yl)-3H-diazirin-3-yl) propanamido)propyl)-7-oxo-7H-benzo[e]perimidine-4-carboxamide (21)

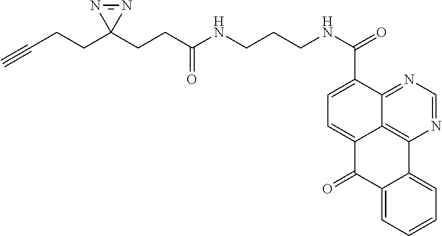 [SMILES: C#CCCC1(CCC(=O)NCCCNC(=O)c2ccc3c4c(ncnc24)-c2ccccc2C3=O)N=N1]

Probe 21 was prepared as described in General Procedure A: Purified by flash chromatography (silica gel, 25% to 100% EtOAc/hexane) to afford 21 as a white solid (0.006 g, 26%).

¹H NMR (500 MHZ, CDCl₃): δ 10.98 (t, J=6.2 Hz, 1H), 9.55 (s, 1H), 9.12 (d, J=7.7 Hz, 1H), 8.94 (d, J=7.8 Hz, 1H), 8.71 (d, J=7.7 Hz, 1H), 8.44 (dd, J=7.7, 1.4 Hz, 1H), 7.89 (td, J=7.6, 1.4 Hz, 1H), 7.81 (td, J=7.5, 1.3 Hz, 1H), 6.64 (s, 1H), 3.72 (q, J=6.3 Hz, 2H), 3.38 (q, J=6.2 Hz, 2H), 2.06-1.99 (m, 5H), 1.93-1.85 (m, 4H), 1.67 (t, J=7.5 Hz, 2H). LCMS (m/z): 481 (M+1). Rₜ: 9.095 min.

### 3-(3-(but-3-yn-1-yl)-3H-diazirin-3-yl)-propanamide (22)

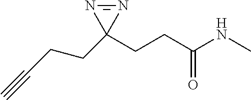

Probe 22 was prepared as described in General Procedure A. Purified by flash chromatography (silica gel, 0% to 30% EtOAc/hexanes) to afford 22 as a colorless oil (0.005 g, 46%).

¹H NMR (500 MHZ, CDCl₃): δ 5.47 (s, 1H), 2.80 (d, J=4.8 Hz, 3H), 2.02 (td, J=7.5, 2.5 Hz, 2H), 1.98 (t, J=2.6 Hz, 1H), 1.94-1.89 (m, 2H), 1.87-1.82 (m, 2H), 1.64 (t, J=7.4 Hz, 2H).

### REFERENCES

Each of the following are incorporated herein by reference for all purposes:
1 Rees, D. C., Congreve, M., Murray, C. W. & Carr, R. Fragment-based lead discovery. Nat Rev Drug Discov 3, 660-672 (2004). https://doi.org: 10.1038/nrd1467
2 Petros, A. M. et al. Discovery of a potent inhibitor of the antiapoptotic protein Bcl-xL from NMR and parallel synthesis. J Med Chem 49, 656-663 (2006). https://doi.org: 10.1021/jm0507532
3 Souers, A. J. et al. ABT-199, a potent and selective BCL-2 inhibitor, achieves antitumor activity while sparing platelets. Nat Med 19, 202-208 (2013). https://doi.org: 10.1038/nm.3048
4 Scott, D. E., Bayly, A. R., Abell, C. & Skidmore, J. Small molecules, big targets: drug discovery faces the protein-protein interaction challenge. Nat Rev Drug Discov 15, 533-550 (2016). https://doi.org: 10.1038/nrd.2016.29
5 Erlanson, D. A. Introduction to fragment-based drug discovery. Top Curr Chem 317, 1-32 (2012). https://doi.org: 10.1007/128_2011_180
6 Li, Q. Application of Fragment-Based Drug Discovery to Versatile Targets. Front Mol Biosci 7, 180 (2020). https://doi.org: 10.3389/fmolb.2020.00180
7 Dang, C. V., Reddy, E. P., Shokat, K. M. & Soucek, L. Drugging the ‘undruggable’ cancer targets. Nat Rev Cancer 17, 502-508 (2017). https://doi.org: 10.1038/nrc.2017.36
8 Parker, C. G. et al. Ligand and Target Discovery by Fragment-Based Screening in Human Cells. Cell 168, 527-541 e529 (2017). https://doi.org: 10.1016/j.cell.2016.12.029
9 Galmozzi, A., Parker, C. G., Kok, B. P., Cravatt, B. F. & Saez, E. Discovery of Modulators of Adipocyte Physiology Using Fully Functionalized Fragments. Methods Mol Biol 1787, 115-127 (2018). https://doi.org: 10.1007/978-1-4939-7847-2_9
10 Vinogradova, E. V. et al. An Activity-Guided Map of Electrophile-Cysteine Interactions in Primary Human T Cells. Cell 182, 1009-1026 e1029 (2020). https://doi.org: 10.1016/j.cell.2020.07.001
11 Li, Z. et al. Design and synthesis of minimalist terminal alkyne-containing diazirine photo-crosslinkers and their incorporation into kinase inhibitors for cell- and tissue-based proteome profiling. Angew Chem Int Ed Engl 52, 8551-8556 (2013). https://doi.org: 10.1002/anie.201300683
12 Welsch, M. E., Snyder, S. A. & Stockwell, B. R. Privileged scaffolds for library design and drug discovery. Curr Opin Chem Biol 14, 347-361 (2010). https://doi.org: 10.1016/j.cbpa.2010.02.018
13 Kombarov, R. et al. BioCores: identification of a drug/natural product-based privileged structural motif for small-molecule lead discovery. Mol Divers 14, 193-200 (2010). https://doi.org: 10.1007/s11030-009-9157-5
14 Tsherniak, A. et al. Defining a Cancer Dependency Map. Cell 170, 564-576 e516 (2017). https://doi.org: 10.1016/j.cell.2017.06.010
15 Canon, J. et al. The clinical KRAS (G12C) inhibitor AMG 510 drives anti-tumour immunity. Nature 575, 217-223 (2019). https://doi.org: 10.1038/s41586-019-1694-1
16 Skoulidis, F. et al. Sotorasib for Lung Cancers with KRAS p.G12C Mutation. N Engl J Med 384, 2371-2381 (2021). https://doi.org: 10.1056/NEJMoa2103695
17 Terai, H. et al. Characterization of DDR2 Inhibitors for the Treatment of DDR2 Mutated Nonsmall Cell Lung Cancer. ACS Chem Biol 10, 2687-2696 (2015). https://doi.org: 10.1021/acschembio.5b00655
18 Weeden, C. E., Solomon, B. & Asselin-Labat, M. L. FGFR1 inhibition in lung squamous cell carcinoma: questions and controversies. Cell Death Discov 1, 15049 (2015). https://doi.org: 10.1038/cddiscovery.2015.49
19 Duan, J., Dixon, S. L., Lowrie, J. F. & Sherman, W. Analysis and comparison of 2D fingerprints: insights into database screening performance using eight fingerprint methods. J Mol Graph Model 29, 157-170 (2010). https://doi.org: 10.1016/j.jmgm.2010.05.008
20 Rishton, G. M. Molecular diversity in the context of leadlikeness: compound properties that enable effective biochemical screening. Curr Opin Chem Biol 12, 340-351 (2008). https://doi.org: 10.1016/j.cbpa.2008.02.008
21 Tan, J. H., Lu, Y. J., Huang, Z. S., Gu, L. Q. & Wu, J. Y. Spectroscopic studies of DNA binding modes of cation-substituted anthrapyrazoles derived from emodin. Eur J Med Chem 42, 1169-1175 (2007). https://doi.org: 10.1016/j.ejmech.2007.02.002
22 Ngai, M. H. et al. Click-based synthesis and proteomic profiling of lipstatin analogues. Chem Commun (Camb) 46, 8335-8337 (2010). https://doi.org: 10.1039/c0cc01276a
23 Jahn, S. C. et al. GSTZ1 expression and chloride concentrations modulate sensitivity of cancer cells to dichloroacetate. Biochim Biophys Acta 1860, 1202-1210 (2016). https://doi.org: 10.1016/j.bbagen.2016.01.024
24 Blackburn, A. C. et al. Deficiency of glutathione transferase zeta causes oxidative stress and activation of antioxidant response pathways. Mol Pharmacol 69, 650-657 (2006). https://doi.org: 10.1124/mol.105.018911
25 Wang, Q. et al. GSTZ1 sensitizes hepatocellular carcinoma cells to sorafenib-induced ferroptosis via inhibition of NRF2/GPX4 axis. Cell Death Dis 12, 426 (2021). https://doi.org: 10.1038/s41419-021-03718-4
26 Townsend, D. M. & Tew, K. D. The role of glutathione-S-transferase in anti-cancer drug resistance. Oncogene 22, 7369-7375 (2003). https://doi.org: 10.1038/sj.onc.1206940
27 Glasauer, A. & Chandel, N. S. Targeting antioxidants for cancer therapy. Biochem Pharmacol 92, 90-101 (2014). https://doi.org: 10.1016/j.bcp.2014.07.017
28 Liao, Y. et al. Synthesis and Antileukemic Activities of Piperlongumine and HDAC Inhibitor Hybrids against Acute Myeloid Leukemia Cells. J Med Chem 59, 7974-7990 (2016). https://doi.org: 10.1021/acs.jmedchem.6b00772
29 Singh, R. R. & Reindl, K. M. Glutathione S-Transferases in Cancer. Antioxidants (Basel) 10 (2021). https://doi.org: 10.3390/antiox10050701
30 Liao, Y. et al. H2O2/Peroxynitrite-Activated Hydroxamic Acid HDAC Inhibitor Prodrugs Show Antileukemic Activities against AML Cells. ACS Med Chem Lett 9, 635-640 (2018). https://doi.org: 10.1021/acsmedchemlett.8b00057
31 Friesner, R. A. et al. Glide: a new approach for rapid, accurate docking and scoring. 1. Method and assessment of docking accuracy. J Med Chem 47, 1739-1749 (2004). https://doi.org: 10.1021/jm0306430
32 Polekhina, G., Board, P. G., Blackburn, A. C. & Parker, M. W. Crystal structure of maleylacetoacetate isomerase/glutathione transferase zeta reveals the molecular basis for its remarkable catalytic promiscuity. Biochemistry 40, 1567-1576 (2001). https://doi.org: 10.1021/bi002249z
33 Tomczak, K., Czerwinska, P. & Wiznerowicz, M. The Cancer Genome Atlas (TCGA): an immeasurable source of knowledge. Contemp Oncol (Pozn) 19, A68-77 (2015). https://doi.org: 10.5114/wo.2014.47136
34 Barrett, T. et al. NCBI GEO: archive for functional genomics data sets-update. Nucleic Acids Res 41, D991-995 (2013). https://doi.org: 10.1093/nar/gks1193
35 Fernandez-Canon, J. M. et al. Maleylacetoacetate isomerase (MAAI/GSTZ)-deficient mice reveal a glutathione-dependent nonenzymatic bypass in tyrosine catabolism Mol Cell Biol 22, 4943-4951 (2002). https://doi.org: 10.1128/MCB.22.13.4943-4951.2002
36 Yang, F. et al. GSTZ1-1 Deficiency Activates NRF2/IGFIR Axis in HCC via Accumulation of Oncometabolite Succinylacetone. EMBO J 38, e101964 (2019). https://doi.org: 10.15252/embj.2019101964
37 Backus, K. M. et al. Proteome-wide covalent ligand discovery in native biological systems. Nature 534, 570-574 (2016). https://doi.org: 10.1038/nature18002
38 Matthews, M. L. et al. Chemoproteomic profiling and discovery of protein electrophiles in human cells. Nat Chem 9, 234-243 (2017). https://doi.org: 10.1038/nchem.2645
39 Hahm, H. S. et al. Global targeting of functional tyrosines using sulfur-triazole exchange chemistry. Nat Chem Biol 16, 150-159 (2020). https://doi.org: 10.1038/s41589-019-0404-5
40 Bar-Peled, L. et al. Chemical Proteomics Identifies Druggable Vulnerabilities in a Genetically Defined Cancer. Cell 171, 696-709 e623 (2017). https://doi.org: 10.1016/j.cell.2017.08.051
41 Solanki, H. S. et al. Cell Type-specific Adaptive Signaling Responses to KRAS (G12C) Inhibition. Clin Cancer Res 27, 2533-2548 (2021). https://doi.org: 10.1158/1078-0432.CCR-20-3872
42 Jiao, D. & Yang, S. Overcoming Resistance to Drugs Targeting KRAS (G12C) Mutation. Innovation (Camb) 1 (2020). https://doi.org: 10.1016/j.xinn.2020.100035
43 Hallin, J. et al. The KRAS (G12C) Inhibitor MRTX849 Provides Insight toward Therapeutic Susceptibility of KRAS-Mutant Cancers in Mouse Models and Patients. Cancer Discov 10, 54-71 (2020). https://doi.org: 10.1158/2159-8290.CD-19-1167
44 Foster, D. A. Metabolic vulnerability of KRAS-driven cancer cells. Mol Cell Oncol 1 (2014). https://doi.org: 10.4161/23723548.2014.963445
45 Crosas-Molist, E. et al. Rho GTPase signaling in cancer progression and dissemination. Physiol Rev 102, 455-510 (2022). https://doi.org: 10.1152/physrev.00045.2020
46 Hajduk, P. J. & Greer, J. A decade of fragment-based drug design: strategic advances and lessons learned. Nat Rev Drug Discov 6, 211-219 (2007). https://doi.org: 10.1038/nrd2220
47 Megger, D. A. et al. Comparison of label-free and label-based strategies for proteome analysis of hepatoma cell lines. Biochim Biophys Acta 1844, 967-976 (2014). https://doi.org: 10.1016/j.bbapap.2013.07.017
48 Stepath, M. et al. Systematic Comparison of Label-Free, SILAC, and TMT Techniques to Study Early Adaption toward Inhibition of EGFR Signaling in the Colorectal Cancer Cell Line DiFi. J Proteome Res 19, 926-937 (2020). https://doi.org: 10.1021/acs.jproteome.9b00701
49 Nogova, L. et al. Evaluation of BGJ398, a Fibroblast Growth Factor Receptor 1-3 Kinase Inhibitor, in Patients With Advanced Solid Tumors Harboring Genetic Alterations in Fibroblast Growth Factor Receptors: Results of a Global Phase I, Dose-Escalation and Dose-Expansion Study. J Clin Oncol 35, 157-165 (2017). https://doi.org: 10.1200/JCO.2016.67.2048
50 Paik, P. K., Pillai, R. N., Lathan, C. S., Velasco, S. A. & Papadimitrakopoulou, V. New Treatment Options in Advanced Squamous Cell Lung Cancer. Am Soc Clin Oncol Educ Book 39, e198-e206 (2019). https://doi.org: 10.1200/EDBK_237829
51 Kitai, H. et al. Epithelial-to-Mesenchymal Transition Defines Feedback Activation of Receptor Tyrosine Kinase Signaling Induced by MEK Inhibition in KRAS-Mutant Lung Cancer. Cancer Discov 6, 754-769 (2016). https://doi.org: 10.1158/2159-8290.CD-15-1377
52 Pacini, L., Jenks, A. D., Lima, N. C. & Huang, P. H. Targeting the Fibroblast

Growth Factor Receptor (FGFR) Family in Lung Cancer. Cells 10 (2021). https://doi.org: 10.3390/cells10051154
53 Hammerman, P. S. et al. Mutations in the DDR2 kinase gene identify a novel therapeutic target in squamous cell lung cancer. Cancer Discov 1, 78-89 (2011). https://doi.org: 10.1158/2159-8274.CD-11-0005
54 Mahajan, K. et al. Ack1 mediated AKT/PKB tyrosine 176 phosphorylation regulates its activation. PLOS One 5, e9646 (2010). https://doi.org: 10.1371/journal.pone.0009646
55 Lu, W., Gong, D., Bar-Sagi, D. & Cole, P. A. Site-specific incorporation of a phosphotyrosine mimetic reveals a role for tyrosine phosphorylation of SHP-2 in cell signaling. Mol Cell 8, 759-769 (2001). https://doi.org: 10.1016/s1097-2765 (01) 00369-0
56 Hopkins, A. L. Network pharmacology: the next paradigm in drug discovery. Nature chemical biology 4, 682-690 (2008). https://doi.org: 10.1038/nchembio.118
57 Palve, V., Liao, Y., Remsing Rix, L. L. & Rix, U. Turning liabilities into opportunities: Off-target based drug repurposing in cancer. Semin Cancer Biol 68, 209-229 (2021). https://doi.org: 10.1016/j.semcancer.2020.02.003
58 Cox, J. & Mann, M. MaxQuant enables high peptide identification rates, individualized p.p.b.-range mass accuracies and proteome-wide protein quantification. Nat Biotechnol 26, 1367-1372 (2008). https://doi.org: 10.1038/nbt.1511
59 MacLean, B. et al. Skyline: an open source document editor for creating and analyzing targeted proteomics experiments. Bioinformatics 26, 966-968 (2010). https://doi.org: 10.1093/bioinformatics/btq054
60 Friesner, R. A. et al. Extra precision glide: docking and scoring incorporating a model of hydrophobic enclosure for protein-ligand complexes. J Med Chem 49, 6177-6196 (2006). https://doi.org: 10.1021/jm0512560
61 Ricci, G. et al. Colorimetric and fluorometric assays of glutathione transferase based on 7-chloro-4-nitrobenzo-2-oxa-1,3-diazole. Anal Biochem 218, 463-465 (1994). https://doi.org: 10.1006/abio.1994.1209
62 Zhou, Y. et al. Metascape provides a biologist-oriented resource for the analysis of systems-level datasets. Nat Commun 10, 1523 (2019). https://doi.org: 10.1038/s41467-019-09234-6

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I 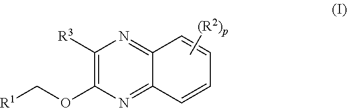
or a pharmaceutically acceptable salt thereof;
wherein:
R¹ is selected from 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more groups independently selected from X as allowed by valency;
R² is independently selected at each occurrence from hydrogen, halo, nitro, cyano, C₁-C₆ alkyl, C₁-C₆ haloalkyl, R^{x}O—(C₀-C₃ alkyl)-, (R^{x}R^{y}N)—(C₀-C₃ alkyl)-, R^{x}O—C(O)—(C₀-C₃ alkyl)-, (R^{x}R^{y}N)—C(O)—(C₀-C₃ alkyl)-, R^{x}O—S(O)₂—(C₀-C₃ alkyl)-, (R^{x}R^{y}N)—S(O)₂—(C₀-C₃ alkyl)-, R^{z}C(O)—(C₀-C₆ alkyl)-, and R^{z}S(O)₂—(C₀-C₃ alkyl)-;
p is 1, 2, 3, or 4;
R³ is selected from C₁-C₆ alkyl, C₁-C₆ haloalkyl, (C₃-C₆ cycloalkyl) (C₀-C₃ alkyl)-, and R^{x}O—(C₀-C₃ alkyl)-;
X is independent selected at each occurrence from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3-to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, (R^{x}R^{y}N)—(C₀-C₃ alkyl)-, R^{x}O—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R⁷C(O)—O—(C₀-C₃ alkyl)-, R^{z}C(O)—(R^{x}N)—(C₀-C₃ alkyl)-, R^{z}S(O)₂-(R^{x}N)—(C₀-C₃ alkyl)-, RIC(O)—(C₀-C₆ alkyl)-, and R^{z}S(O)₂-(C₀-C₃ alkyl)-, each of which may be optionally substituted with one or more groups independently selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5-to 10-membered monocyclic or bicyclic heteroaryl)-(C₀-C₃ alkyl)-, each of which may be optionally substituted with one or more groups independently selected from Y as allowed by valency;
R^{z} is independently selected at each occurrence from hydrogen, halo, C₁-C₀alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5-to 10-membered monocyclic or bicyclic heteroaryl)-(C₀-C₃ alkyl)-, —OR^{x}, —SR^{x}, and —NR^{x}R^{y}, each of which may be optionally substituted with one or more groups independently selected from Y as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. The compound of claim 1, wherein R¹ is 3- to 8-membered monocyclic or bicyclic heterocycle optionally substituted with one or more groups independently selected from X as allowed by valency.

3. The compound of claim 2, wherein R¹ is selected from:

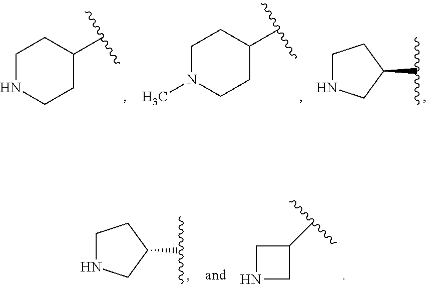

4. The compound of claim 1, wherein R¹ is 6- to 10-membered monocyclic or bicyclic aryl optionally substituted with one or more groups independently selected from X as allowed by valency.

5. The compound of claim 4, wherein R¹ is

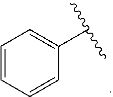 [SMILES: *c1ccccc1]

6. The compound of claim 1, wherein R¹ is 5- to 10-membered monocyclic or bicyclic heteroaryl.

7. The compound of claim 6, wherein R¹ is selected from:

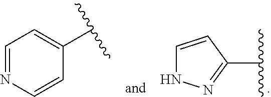 [SMILES: *c1ccncc1 ; *c1cc[nH]n1]

8. The compound of claim 1, wherein R³ is C₁-C₆ alkyl.

9. The compound of claim 8, wherein R³ is selected from methyl, ethyl, isopropyl, and tert-butyl.

10. The compound of claim 1, wherein R³ is C₁-C₆ haloalkyl.

11. The compound of claim 10, wherein R³ is selected from —CF₃ and —CH₂CF₃.

12. The compound of claim 1, wherein R³ is (C₃-C₆ cycloalkyl) (C₀-C₃ alkyl)-.

13. The compound of claim 12, wherein R³ is selected from:

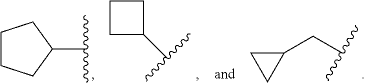

14. The compound of claim 1, wherein R³ is R^{x}O—(C₀-C₃ alkyl)-.

15. The compound of claim 14, wherein R³ is —CH₂OCH₃.

16. The compound of claim 1, wherein R² is independently selected at each occurrence from hydrogen, chloro, bromo, iodo, —OH, —OCH₃, —CH₃, tert-butyl, —CF₃, —NH₂, —N(CH₃)₂, nitro, cyano, —S(O)₂—CH₃, —S(O)₂—NH₂, and —C(O)—NH₂.

17. The compound of claim 1,
wherein 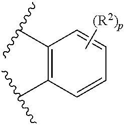
is selected from:

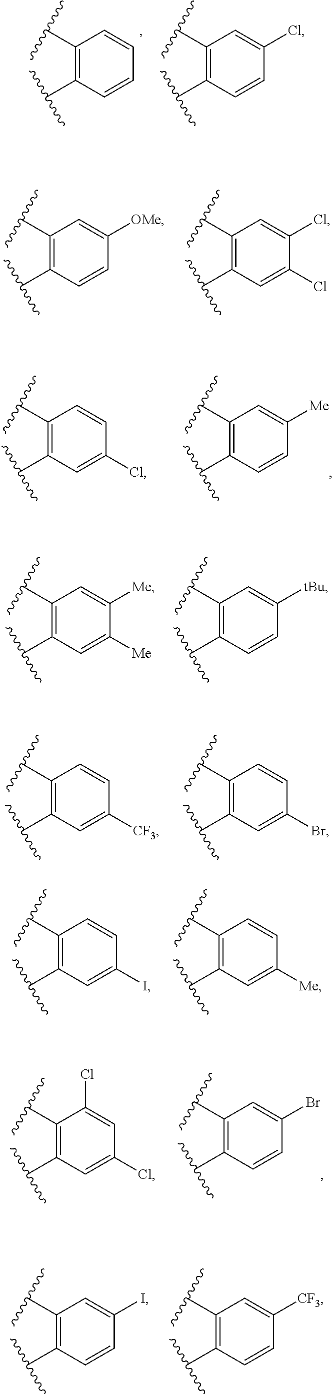

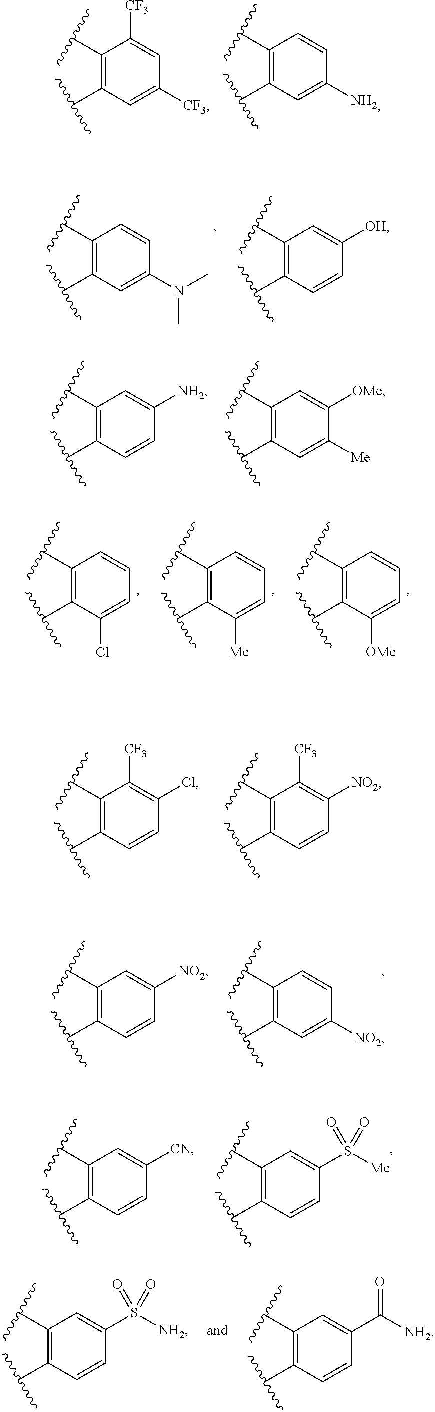

18. The compound of claim 1, wherein the compound is selected from: 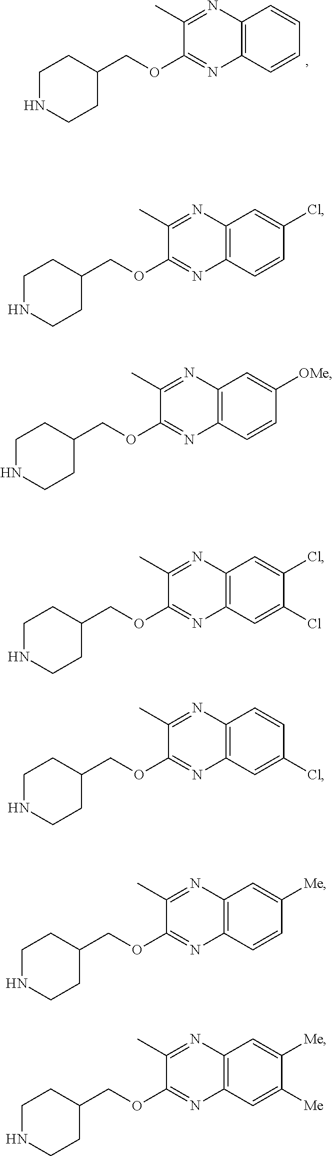 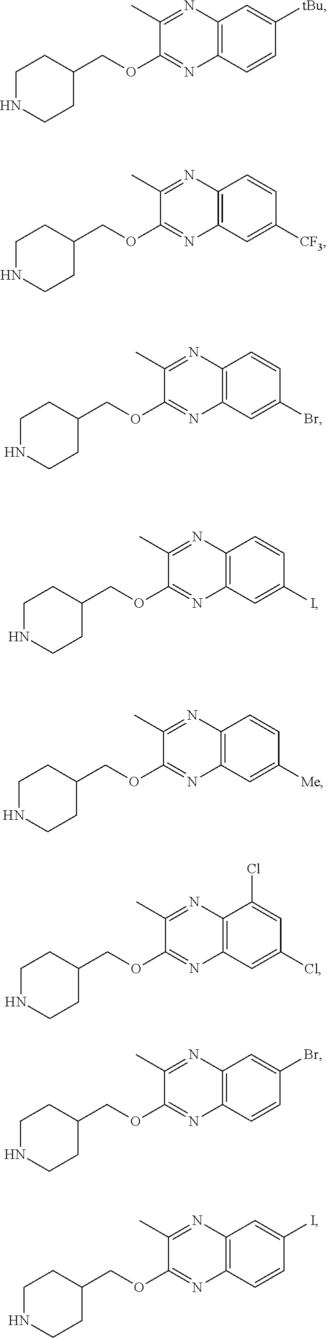 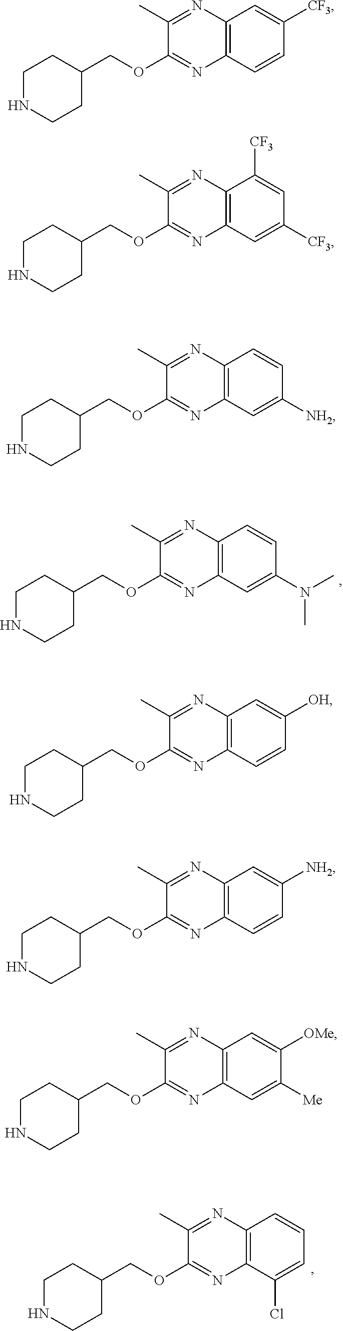 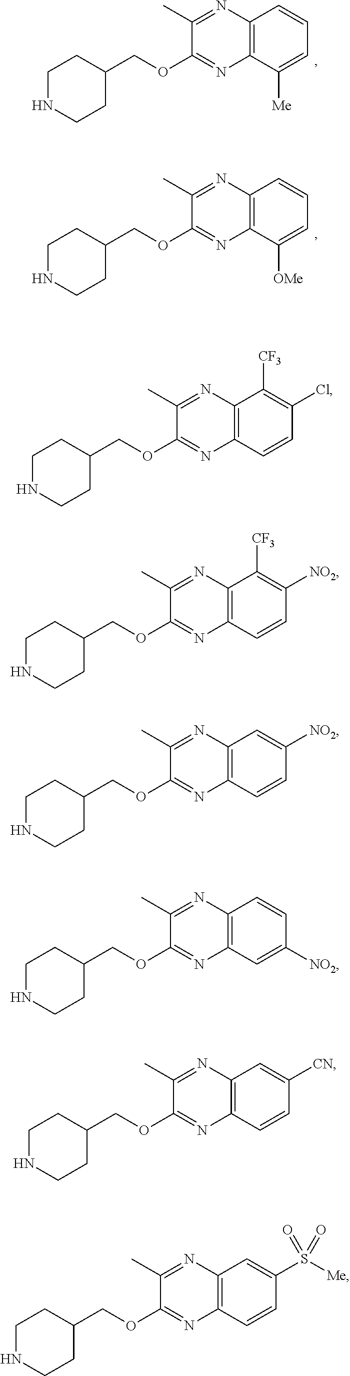 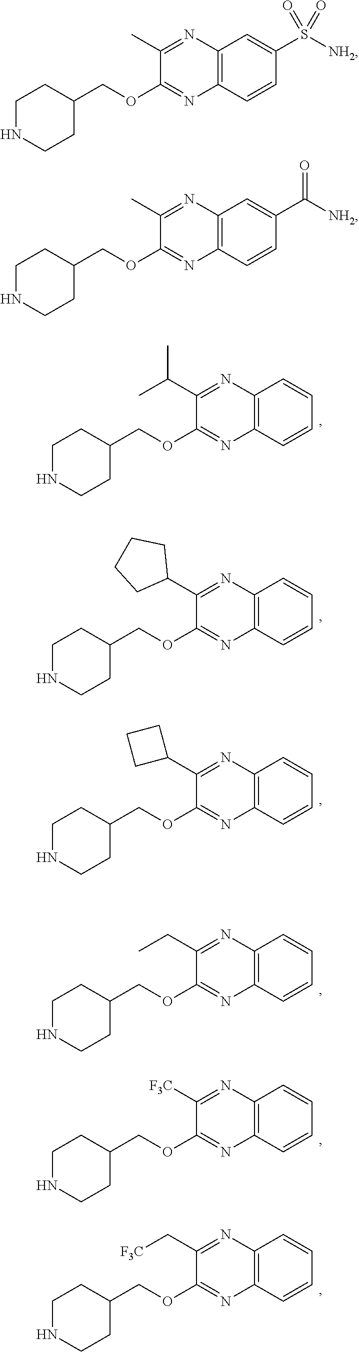 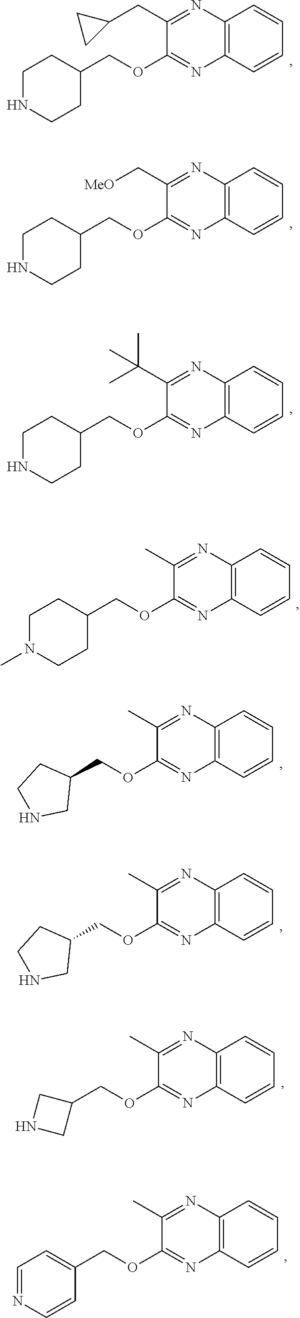 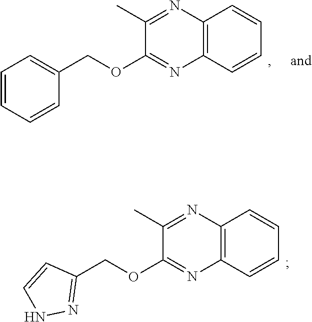 [SMILES: Cc1nc2ccccc2nc1OCc1ccccc1 ; Cc1nc2ccccc2nc1OCc1cc[nH]n1]
or a pharmaceutically acceptable salt thereof.

19. A pharmaceutical composition comprising a compound of claim 1, or a pharmaceutically acceptable salt thereof, and a pharmaceutically acceptable carrier or excipient.

20. A method of treating cancer in a subject in need thereof comprising administering to the subject a therapeutically effective amount of a compound of claim 1, or a pharmaceutically acceptable salt thereof.

21-24. (canceled)
